(19) United States
(12) Patent Application Publication
(10) Pub. No.: US 2012/0318971 A1
(43) Pub. Date: Dec. 20, 2012

(54) ANALYSIS OF TOTAL HOMOCYSTEINE AND METHYLMALONIC ACID IN PLASMA BY LC-MS/MS FROM A PLASMA SEPARATOR DEVICE (PSD)

(75) Inventors: Teodoro G. Bottiglieri, Dallas, TX (US); Erland Arning, Fort Worth, TX (US)

(73) Assignee: BAYLOR RESEARCH INSTITUTE, Dallas, TX (US)

(21) Appl. No.: 13/495,894

(22) Filed: Jun. 13, 2012

Related U.S. Application Data
(60) Provisional application No. 61/497,647, filed on Jun. 16, 2011.

(51) Int. Cl. H01J 49/26; G01N 1/28

(52) U.S. Cl. 250/282; 250/288; 73/863.21

(57) ABSTRACT

The present invention provides a method of diagnosing multiple disorders and distinguishing there between using a plasma sample obtained from a plasma separator device and analyzing the plasma sample using an LC-MS/MS to detect at least two analyte levels in the plasma sample to diagnose one or more disorders.

### CROSS-REFERENCE TO RELATED APPLICATIONS

This application claims priority to U.S. Provisional Application Ser. No. 61/497,647 filed Jun. 16, 2011, the entire contents of which are incorporated herein by reference.

### TECHNICAL FIELD OF THE INVENTION

The present invention relates in general to platforms, devices and methods useful for analytical assays especially concerned with determining the presence of one or more analytes in small volumes of whole blood, although it is not so limited.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with determining the presence of one or more analytes in a small blood sample.

Currently, it is common practice to detect or quantify distinct analytes using distinct detection or quantification techniques. For example, enzyme assays, immunoassays, chemical colorimetric assays, fluorescence labeling and measurement, chemiluminescent labeling and measurement, and electrochemiluminescent labeling and measurement, are a few exemplary well-known analytical techniques that may be used to detect the presence of various analytes. Many of these techniques are performed on a test strip or cartridge.

For example, U.S. Pat. No. 4,940,658, entitled, “Assay for Sulfhydryl Amino Acids and Methods for Detecting and Distinguishing Cobalamin and Folic Acid Deficiency,” discloses a method for determining levels of sulfhydryl amino acids, particularly total homocysteine levels in samples of body tissue from warm-blooded animals, methods of detecting cobalamin and folic acid deficiency using an assay for total homocysteine levels, and methods for distinguishing cobalamin from folic acid deficiency using an assay for total homocysteine levels in conjunction with an assay for methylmalonic acid.

U.S. Pat. No. 5,435,970, entitled, “Device for Analysis for Constituents in Biological Fluids,” discloses a device for separating blood cells from biological fluids, preferably plasma from whole blood.

U.S. Pat. No. 7,407,742, entitled, “Plasma or Serum Separator, Plasma or Serum Sampling Method, Plasma or Serum Separating Method, Test Carrier and Glass Fiber,” disclose a plasma or serum separator and a plasma or serum sampling method capable of isolating plasma or serum with good efficiency from a small amount of blood without using a centrifuge and without causing leakage of a blood cell component or hemolysis, and in addition, capable of isolating and collecting plasma or serum from a whole blood test sample in a short time with simplicity in a blood test in the scene of medical care requiring an instant treatment any time such as an emergency test, home-use test or the like.

U.S. patent application Ser. No. 12/867,335, entitled, “Apparatus for the Separation of Plasma,” discloses an apparatus for separating blood, more particularly an apparatus for absorbing blood and separating blood components, e.g., blood plasma, as a sample liquid. Said apparatus comprises a feeding device for absorbing the blood, a device for separating blood components as a sample liquid, a duct which preferably absorbs the sample liquid exclusively by means of capillary forces, and a device for filling the duct with sample liquid in an inlet or feeding zone of the duct. The separating device, in particular a membrane, is curved, especially convexly shaped, and the apex of said curved, especially convex shape projects into the filling device.

### SUMMARY OF THE INVENTION

The present invention provides a method of diagnosing one or more disorders and distinguishing there between using a single dried blood sample by obtaining a plasma sample from a plasma separator device and analyzing the plasma sample using a liquid-chromatography-tandem-mass spectrometer (LC-MS/MS) to detect at least two analyte levels in the plasma sample to diagnose one or more disorders, wherein at least two analyte levels are selected from total homocysteine, methylmalonic acid, S-adenosylmethionine, S-adenosylhomocysteine, betaine, choline, asymmetric dimethylarginine, symmetric dimethylarginine, creatinine, amino acids, glutathione, Vitamin D, Vitamin B1 (thiamine), Vitamin B2 (riboflavin), Vitamin B3 (niacin), Vitamin B4 (adenine), Vitamin B5 (pantothenic acid), Vitamin B6 (pyridoxine), Vitamin B7 (biotin), Vitamin B12, folate, or iron. The plasma separator device includes a removable holding member to cover a semi-permeable blood separation member, a blood introducing portion formed in a portion of the holding member and in communication with the semi-permeable blood separation member, a removable plasma sample collection reservoir in communication with the semi-permeable blood separator member, wherein a whole blood sample is deposited on the blood introducing portion and separated by the semi-permeable blood separator member and a plasma sample is collected in the removable plasma sample collection reservoir, and a base in communication with the removable plasma sample collection reservoir. The removable plasma sample collection reservoir may be removed from the base and the plasma sample can be isolated from the removable plasma sample collection reservoir. In addition, a white blood cell sample can be isolated from the removable holding member. The present invention can analyze two or more analytes and may include 1, 2, 3, 4, 5, 6, 7, 8, 9, 10, 11, 12, 13, 14 or more additional analyte levels selected from total homocysteine, methylmalonic acid, S-adenosylmethionine, S-adenosylhomocysteine, betaine, choline, asymmetric dimethylarginine, symmetric dimethylarginine, creatinine, amino acids, glutathione, Vitamin D, Vitamin B1 (thiamine), Vitamin B2 (riboflavin), Vitamin B3 (niacin), Vitamin B4 (adenine), Vitamin B5 (pantothenic acid), Vitamin B6 (pyridoxine), Vitamin B7 (biotin), Vitamin B12, folate, and/or iron. The plasma separator device is received by mail or any other method of delivery. In one aspect, the one or more multiple disorders are selected from disease is selected from at least one of a nutritional disease or disorder, a hematological disease, a psychiatric disease, a neurological disease, a vascular disease, a peripheral disease, a cardiovascular disease, a cerebrovascular disease, a genetic metabolic disorder, a renal insufficiency, an Argininemia, an Argininosuccinic Aciduria, a Carbamoylphosphate Synthetase Deficiency1, a Citrullinemia, a Homocystinuria, a Hypermethioninemia, a Hyperammonemia, a Hyperornithinemia, a Homocitrullinuria, a Maple Syrup Urine Disease (MSUD), a Phenylketonuria (Classical Hyperphenylalaninemia/Biopterin Cofactor Deficiencies), a Tyrosinemia, a Cystathionine beta-synthease deficiency (elevated Homocysteine and methionine); a Methylenetetrahydrofolate reductase deficiency (MTHFR, elevated homocsyteine, low methionine); or a Methylmalonic Acidemia.

The present invention also provides a method of diagnosing a disease by obtaining a plasma sample from a plasma separator device, wherein the plasma separator device comprises a removable holding member to cover a semi-permeable blood separation member, a blood introducing portion formed in a portion of the holding member and in communication with the semi-permeable blood separation member, a removable plasma sample collection reservoir in communication with the semi-permeable blood separator member, wherein a whole blood sample is deposited on the blood introducing portion and separated by the semi-permeable blood separator member and a plasma sample is collected in the removable plasma sample collection reservoir, and a base in communication with the removable plasma sample collection reservoir; and analyzing the plasma sample using an LC-MS/MS to detect at least two analyte levels in the plasma sample to diagnose one or more disorders, wherein at least two analyte levels are selected from total homocysteine, methylmalonic acid, S-adenosylmethionine, S-adenosylhomocysteine, betaine, choline, asymmetric dimethylarginine, symmetric dimethylarginine, creatinine, amino acids, glutathione, Vitamin D, Vitamin B1 (thiamine), Vitamin B2 (riboflavin), Vitamin B3 (niacin), Vitamin B4 (adenine), Vitamin B5 (pantothenic acid), Vitamin B6 (pyridoxine), Vitamin B7 (biotin), Vitamin B12, folate, or iron. The present invention may analyze 1, 2, 3, 4, 5, 6, 7, 8, 9, 10, 11, 12, 13, 14 or more additional analyte levels selected from total homocysteine, methylmalonic acid, s-adenosylhomocysteine, betaine, choline, asymmetric dimethylarginine, Symmetric dimethylarginine, creatinine, amino acids, glutathione, Vitamin D, Vitamin B1 (thiamine), Vitamin B2 (riboflavin), Vitamin B3 (niacin), Vitamin B4 (adenine), Vitamin B5 (pantothenic acid), Vitamin B6 (pyridoxine), Vitamin B7 (biotin), Vitamin B12, folate, or iron and these are used to determine 1, 2, 3, 4, 5, 6, 7, 8, 9, 10, 11, 12, 13, 14 or more diseases. In one aspect, the disease is selected from at least one of a nutritional disease or disorder, a hematological disease, a psychiatric disease, a neurological disease, a vascular disease, a peripheral disease, a cardiovascular disease, a cerebrovascular disease, a genetic metabolic disorder, a renal insufficiency, an Argininemia, an Argininosuccinic Aciduria, a Carbamoylphosphate Synthetase Deficiency1, a Citrullinemia, a Homocystinuria, a Hypermethioninemia, a Hyperammonemia, a Hyperornithinemia, a Homocitrullinuria, a Maple Syrup Urine Disease, a Phenylketonuria (Classical Hyperphenylalaninemia/Biopterin Cofactor Deficiencies), a Tyrosinemia, a Cystathionine beta-synthetase deficiency (elevated Homocysteine and methionine); a Methylenetetrahydrofolate reductase deficiency (MTHFR, elevated homocysteine, low methionine); or a Methylmalonic Acidemia.

Also disclosed is a method of diagnosing a vascular risk factor by obtaining a plasma sample from a plasma separator device, wherein the plasma separator device comprises a removable holding member to cover a semi-permeable blood separation member, a blood introducing portion formed in a portion of the holding member and in communication with the semi-permeable blood separation member, a removable plasma sample collection reservoir in communication with the semi-permeable blood separator member, wherein a whole blood sample is deposited on the blood introducing portion and separated by the semi-permeable blood separator member and a plasma sample is collected in the removable plasma sample collection reservoir, and a base in communication with the removable plasma sample collection reservoir; and analyzing the plasma sample using an LC-MS/MS to detect at least two analyte levels in the plasma sample to diagnose a vascular risk factor, wherein the at least two analyte levels are selected from total homocysteine, S-adenosylmethionine, S-adenosylhomocysteine, asymmetric dimethylarginine, and symmetric dimethylarginine. The present invention can analyze 1 or 2 additional analyte levels selected from total homocysteine, S-adenosylmethionine, S-adenosylhomocysteine, asymmetric dimethylarginine, and symmetric dimethylarginine, or even more analytes and vascular diseases or conditions.

The present invention discloses a method of diagnosing a genetic metabolic disorder by obtaining a plasma sample from a plasma separator device, wherein the plasma separator device comprises a removable holding member to cover a semi-permeable blood separation member, a blood introducing portion formed in a portion of the holding member and in communication with the semi-permeable blood separation member, a removable plasma sample collection reservoir in communication with the semi-permeable blood separator member, wherein a whole blood sample is deposited on the blood introducing portion and separated by the semi-permeable blood separator member and a plasma sample is collected in the removable plasma sample collection reservoir, and a base in communication with the removable plasma sample collection reservoir; and analyzing the plasma sample using an LC-MS/MS to detect at least two analyte levels in the plasma sample to diagnose a genetic metabolic disorder, wherein at least two analyte levels are selected from total Homocysteine, Methionine, S-adenosylmethionine, S-adenosylhomocysteine, and Amino Acids. The present invention can analyze 1, 2, or 3 additional analyte levels selected from total Homocysteine, Methionine, S-adenosylmethionine, S-adenosylhomocysteine, and Amino Acids.

The present invention provides a method of diagnosing a renal insufficiency by obtaining a plasma sample from a plasma separator device, wherein the plasma separator device comprises a removable holding member to cover a semi-permeable blood separation member, a blood introducing portion formed in a portion of the holding member and in communication with the semi-permeable blood separation member, a removable plasma sample collection reservoir in communication with the semi-permeable blood separator member, wherein a whole blood sample is deposited on the blood introducing portion and separated by the semi-permeable blood separator member and a plasma sample is collected in the removable plasma sample collection reservoir, and a base in communication with the removable plasma sample collection reservoir; and analyzing the plasma sample using an LC-MS/MS to detect at least two analyte levels in the plasma sample to diagnose a renal insufficiency, wherein at least two analyte levels are selected from S-adenosylhomocysteine, asymmetric dimethylarginine, symmetric dimethylarginine, and creatinine. The present invention can analyze 1, 2, or 3 additional analyte levels selected from S-adenosylhomocysteine, Asymmetric dimethylarginine, Symmetric dimethylarginine, and creatinine.

The present invention provides a method for detecting a deficiency of cobalamin, folate, or both and distinguishing there between by obtaining a plasma sample from a plasma separator device, and analyzing the plasma sample using an LC-MS/MS to detect the presence of elevated levels of total homocysteine and methylmalonic acid, wherein elevated levels of total homocysteine and methylmalonic acid may indicate cobalamin deficiency, and elevated levels of total homocysteine combined with normal levels of methylmalonic acid may indicate folic acid deficiency to diagnose deficiency of cobalamin, folate, or both. The plasma separator device includes a removable holding member to cover a semi-permeable blood separation member, a blood introducing portion formed in a portion of the holding member and in communication with the semi-permeable blood separation member, a removable plasma sample collection reservoir in communication with the semi-permeable blood separator member, wherein a whole blood sample is deposited on the blood introducing portion and separated by the semi-permeable blood separator member and a plasma sample is collected in the removable plasma sample collection reservoir, and a base in communication with the removable plasma sample collection reservoir.

The present invention also provides a method of monitoring a drug level in a subject in a clinical trial by (a) providing a subject involved in a clinical trial; (b) obtaining a plasma separator device from the subject; (c) obtaining a plasma sample from the plasma separator device; (d) analyzing the plasma sample using an LC-MS/MS to detect at least two analyte levels in the plasma sample, wherein at least two analyte levels are selected from total Homocysteine (tHcy), Methylmalonic acid (MMA), S-adenosylmethionine, S-adenosylhomocysteine (SAH), Betaine, Choline, Asymmetric dimethylarginine (ADMA), Symmetric dimethylarginine (SDMA), creatinine, Amino Acids (up to and including a full screen 42 compounds), glutathione, Vitamin D, Vitamin B1 (thiamine), Vitamin B2 (riboflavin), Vitamin B3 (niacin), Vitamin B4 (adenine), Vitamin B5 (pantothenic acid), Vitamin B6 (pyridoxine), Vitamin B7 (biotin), Vitamin B12, folate, or iron; (e) providing an agent to the subject; (f) analyzing the blood plasma sample using an LC-MS/MS to detect a agent level; and (g) repeating steps (a) to (f). The plasma separator device may include a removable holding member to cover a semi-permeable blood separation member, a blood introducing portion formed in a portion of the holding member and in communication with the semi-permeable blood separation member, a removable plasma sample collection reservoir in communication with the semi-permeable blood separator member, wherein a whole blood sample is deposited on the blood introducing portion and separated by the semi-permeable blood separator member and a plasma sample is collected in the removable plasma sample collection reservoir, and a base in communication with the removable plasma sample collection reservoir. In one aspect, the clinical trial is for a nutritional disorder, a hematological disease, a psychiatric disease, a neurological disease, a vascular disease, a peripheral disease, a cardiovascular disease, a cerebrovascular disease, a genetic metabolic disorder, or a renal insufficiency. In another aspect, the clinical trial is pre-clinical trial and the subject is a cat, a dog, a goat, a non-human primate, a mouse, a pig, or a rat. In another aspect, the clinical trial is clinical drug trial and the subject is a human.

The present invention provides a system for diagnosing multiple disorders and distinguishing there between from a single dried blood sample including a plasma separator and an LC-MS/MS system to detect at least two analyte levels in the plasma sample to diagnose multiple disorders and distinguishing there between, wherein at least two analyte levels are selected from total Homocysteine, Methylmalonic acid (MMA), S-adenosylhomocysteine (SAH), Betaine, Choline, Asymmetric dimethylarginine (ADMA), Symmetric dimethylarginine (SDMA), creatinine, Amino Acids (full screen 42 compounds), Vitamin D, Vitamin B1 (thiamine), Vitamin B2 (riboflavin), Vitamin B3 (niacin), Vitamin B4 (adenine), Vitamin B5 (pantothenic acid), Vitamin B6 (pyridoxine), Vitamin B7 (biotin), Vitamin B12, folate, or iron. The plasma separator includes a removable holding member to cover a semi-permeable blood separation member, a blood introducing portion formed in a portion of the holding member and in communication with the semi-permeable blood separation member, a removable plasma sample collection reservoir in communication with the semi-permeable blood separator member, wherein a whole blood sample deposited on the blood introducing portion is separated by the semi-permeable blood separator member and a plasma sample is collected in the removable plasma sample collection reservoir, and a base in communication with the removable plasma sample collection reservoir.

A method of diagnosing a metabolic disorder is also disclosed to include obtaining a plasma sample from a plasma separator device, wherein the plasma separator device comprises a removable holding member to cover a semi-permeable blood separation member, a blood introducing portion formed in a portion of the holding member and in communication with the semi-permeable blood separation member, a removable plasma sample collection reservoir in communication with the semi-permeable blood separator member, wherein a whole blood sample is deposited on the blood introducing portion and separated by the semi-permeable blood separator member and a plasma sample is collected in the removable plasma sample collection reservoir, and a base in communication with the removable plasma sample collection reservoir; and analyzing the plasma sample using an LC-MS/MS to detect at least two analyte levels in the plasma sample to diagnose a metabolic disorder, wherein at least two analyte levels are selected from total Homocysteine (tHcy), Methylmalonic acid (MMA), S-adenosylmethionine (SAM), S-adenosylhomocysteine (SAH), Betaine, Choline, Asymmetric dimethylarginine (ADMA), Symmetric dimethylarginine (SDMA), creatinine, Amino Acids (up to and including a full screen 42 compounds), glutathione, Vitamin D, Vitamin B1 (thiamine), Vitamin B2 (riboflavin), Vitamin B3 (niacin), Vitamin B4 (adenine), Vitamin B5 (pantothenic acid), Vitamin B6 (pyridoxine), and Vitamin B7 (biotin). In addition, 1, 2, 3, 4, 5, 6, 7, 8, 9, 10, 11, 12, 13, or 14 additional analyte levels selected from total homocysteine, methylmalonic acid, s-adenosylhomocysteine, betaine, choline, asymmetric dimethylarginine, Symmetric dimethylarginine, creatinine, an amino acids, Vitamin D, Vitamin B1 (thiamine), Vitamin B2 (riboflavin), Vitamin B3 (niacin), Vitamin B4 (adenine), Vitamin B5 (pantothenic acid), Vitamin B6 (pyridoxine), Vitamin B7 (biotin), Vitamin B12, folate, or iron may be analyzed.

The present invention includes a method of multiplex sample analysis from a single dried blood sample by obtaining a blood plasma sample from a plasma separator device, wherein the plasma separator device comprises a removable holding member to cover a semi-permeable blood separation member, a blood introducing portion formed in a portion of the holding member and in communication with the semi-permeable blood separation member, a removable plasma sample collection reservoir in communication with the semi-permeable blood separator member, wherein a whole blood sample is deposited on the blood introducing portion and separated by the semi-permeable blood separator member and the blood plasma sample is collected in the removable plasma sample collection reservoir, and a base in communication with the removable plasma sample collection reservoir; labeling one or more components of the plasma sample and analyzing the plasma sample using an Liquid-chromatography-tandem-mass spectrometer (LC-MS/MS) to detect the one or more components in the plasma sample. The multiplex analysis of the present invention can combine quantifications based on ratios of MS/MS ions deriving from unlabeled and labeled precursors (ex: ICAT) co-fragmented in the same MS/MS (ex: iTRAQ) scan. In addition, the present invention may be used to detect metabolic disorders, sickle cell disorders, HIV, malarial infections, and other disorders and infections. As such, the present invention provides a multi-analyte plasma separator device and methods for “targeted” and “non-targeted” proteomic analysis from a single dried blood sample.

Yet another embodiment of the present invention includes a blood plasma separator comprising: a removable holding member to cover a semi-permeable blood separation member, a blood introducing portion formed in a portion of the holding member and in communication with the semi-permeable blood separation member, a removable blood plasma sample collection reservoir in communication with the semi-permeable blood separator member, wherein a whole blood sample deposited on the blood introducing portion is separated by the semi-permeable blood separator member and the blood plasma sample is collected in the removable blood plasma sample collection reservoir, and a base in communication with the removable blood plasma sample collection reservoir. Yet another embodiment of the present invention includes a method of monitoring a drug level in a subject comprising the steps of: (a) obtaining a blood plasma separator device from the subject; (b) obtaining a blood plasma sample from the blood plasma separator device, wherein the blood plasma separator device comprises a removable holding member to cover a semi-permeable blood separation member, a blood introducing portion formed in a portion of the holding member and in communication with the semi-permeable blood separation member, a removable blood plasma sample collection reservoir in communication with the semi-permeable blood separator member, wherein a whole blood sample is deposited on the blood introducing portion and separated by the semi-permeable blood separator member and the blood plasma sample is collected in the removable blood plasma sample collection reservoir, and a base in communication with the removable blood plasma sample collection reservoir; (c) analyzing the blood plasma sample using a LC-MS/MS to detect at least two analyte levels in the plasma sample, wherein the at least two analyte levels are selected from total Homocysteine (tHcy), Methylmalonic acid (MMA), S-adenosylhomocysteine (SAH), Betaine, Choline, Asymmetric dimethylarginine (ADMA), Symmetric dimethylarginine (SDMA), creatinine, Amino Acids (up to and including a full screen 42 compounds), glutathione, phenylalanine, Vitamin D, Vitamin B1 (thiamine), Vitamin B2 (riboflavin), Vitamin B3 (niacin), Vitamin B4 (adenine), Vitamin B5 (pantothenic acid), Vitamin B6 (pyridoxine), Vitamin B7 (biotin), Vitamin B12, folate, or iron; (d) providing an agent to the subject; e) analyzing the blood plasma sample using an LC-MS/MS to detect the level of the agent; and (f) optionally repeating steps (a) to (e), if necessary. In one aspect, the disease is selected from at least one of a nutritional disorder, a hematological disease, a psychiatric disease, a neurological disease, a vascular disease, a peripheral disease, a cardiovascular disease, a cerebrovascular disease, a genetic metabolic disorder, a renal insufficiency, an Argininemia, an Argininosuccinic Aciduria, a Carbamoylphosphate Synthetase Deficiency1, a Citrullinemia, a Homocystinuria, a Hypermethioninemia, a Hyperammonemia, a Hyperornithinemia, a Homocitrullinuria, a Maple Syrup Urine Disease, a Phenylketonuria, a Tyrosinemia, a Cystathionine beta-synthease deficiency, a Methylenetetrahydrofolate reductase deficiency, or a Methylmalonic Acidemia.

### BRIEF DESCRIPTION OF THE DRAWINGS

For a more complete understanding of the features and advantages of the present invention, reference is now made to the detailed description of the invention along with the accompanying figures and in which:

FIG. 1 is an image of a LC/MS/MS system.

FIGS. 2A and 2B are the data and the plot that correlates tHcy testing in plasma from blood draw versus finger stick plasma separator device (PSD).

FIG. 3 is an image of a plasma separator device.

FIGS. 4A and 4B are images of the Multiple reaction monitoring (MRM) plot of the liquid-chromatography-tandem-mass spectrometry (LC-MS/MS) determination of Plasma tHcy for a standard (FIG. 4A) and the sample (FIG. 4B).

FIGS. 5A-5D are tables demonstrating recovery from a plasma sample and a PSD sample for subject 1 and 2 and includes expected and observed concentrations for tHcy and amount of spiked standard recovered.

FIGS. 6A-6B are the data and the plot that correlate MMA testing in plasma from plasma versus plasma spotted on PSD.

FIG. 7 shows one of the procedures for sample extraction and analysis of the present invention.

FIG. 8 is a graph that shows the stability of tHcy on PSD at room temperature.

FIG. 9 is a graph that shows the volume-dependence for PSD tHcy.

FIG. 10 is a graph that shows the results from simultaneous venepuncture and PSD collection in subjects with ESRD.

FIG. 11 is a graph that shows the correlation for Tyrosine in plasma versus PSD.

FIG. 12 is a graph that shows the correlation for Phenylalanine in plasma versus PSD.

FIG. 13 is a graph that shows the correlation for ADMA in plasma versus PSD.

FIG. 14 is a graph that shows the correlation for SDMA in plasma versus PSD.

FIG. 15 is a graph that shows the correlation for Arginine in plasma versus PSD.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, “inorganic molecule” refers to a molecule that does not contain hydrocarbon group(s).

As used herein, “organic molecule” refers to a molecule that contains hydrocarbon group(s).

As used herein, “vitamin” refers to a trace organic substance required in certain biological species.

As used herein, “biomolecule” refers to an organic compound normally present as an essential component of living organisms.

As used herein, “lipid” refers to water-insoluble, oily or greasy organic substances that are extractable from cells and tissues by nonpolar solvents, such as chloroform or ether.

As used herein, “Homocysteine” (Hcy) refers to a compound with the following molecular formula: HSCH₂ CH₂ CH(NH₂)COOH. Biologically, Hcy is produced by demethylation of methionine and is an intermediate in the biosynthesis of cysteine from methionine. The term “Hcy” encompasses free Hcy (in the reduced form) and conjugated Hcy (in the oxidized form). Hcy can conjugate with proteins, peptides, itself or other thiols through disulfide bond.

As used herein, “serum” refers to the fluid portion of the blood obtained after removal of the fibrin clot and blood cells, distinguished from the plasma in circulating blood.

As used herein, “plasma” refers to the fluid, noncellular portion of the blood, distinguished from the serum obtained after coagulation.

As used herein, “substantially pure” refers to a sufficiently homogeneous composition that is free of readily detectable impurities as determined by standard methods of analysis, such as thin layer chromatography, gel electrophoresis and high performance liquid chromatography, used by those of skill in the art to assess such purity, or sufficiently pure such that further purification would not detectably alter the physical and chemical properties, such as enzymatic and biological activities, of the substance.

As used herein, “sample” refers to anything that may contain an analyte for which an analyte assay is desired. The sample may be a biological sample, such as a biological fluid supernatant, e.g., urine, blood, plasma, serum, saliva, semen, stool, sputum, cerebral spinal fluid, tears, mucus, amniotic fluid or the like.

As used herein, “Multiplex assay” refers to a type of procedure that simultaneously measures multiple analytes (dozens or more) in a single assay and is distinguished from procedures that measure one or a few analytes at a time. Multiplex assays are widely used to detect or to assay a given class of molecules within a biological sample, to determine the effect of a treatment.

As used herein, “analyte” refers to any molecule(s), including biological macromolecules and small molecules, elements or ions, organic or inorganic molecules, ligands, anti-ligands and other species that can be detected using the present invention. The methods, systems and separator of the present invention can be used to assay an analyte. For example, inorganic molecule may be an inorganic ion such as a sodium, a potassium, a magnesium, a calcium, a chlorine, an iron, a copper, a zinc, a manganese, a cobalt, an iodine, a molybdenum, a vanadium, a nickel, a chromium, a fluorine, a silicon, a tin, a boron or an arsenic ion. Organic molecule to be assayed may be an amino acid, a peptide, a nucleoside, a nucleotide, an oligonucleotide, a vitamin, a monosaccharide, an oligosaccharide, a lipid or a protein. The following abbreviations are used for the various analytes: Homocysteine (tHcy), Methylmalonic acid (MMA), Methionine, S-adenosylmethionine (SAM), S-adenosylhomocysteine (SAH), Asymmetric dimethylarginine (ADMA), Symmetric dimethylarginine (SDMA), Vitamin B1 (thiamine), Vitamin B2 (riboflavin), Vitamin B3 (niacin), Vitamin B4 (adenine), Vitamin B5 (pantothenic acid), Vitamin B6 (pyridoxine), Vitamin B7 (biotin), Vitamin B12, folate, or iron.

Depending on the analyte(s) tested the present invention can be used to determine, identify and/or diagnose a number of disorders including, but not limited to, nutritional deficiencies, disorders or diseases that include clinical symptoms and/or diseases that are hematological, psychiatric and/or neurological. Other diseases include vascular risk factors or diseases or disorders that include vascular disease, peripheral disease, cardiovascular disease, and/or cerebrovascular disease. The present invention can also identify genetic metabolic disorders, and/or renal insufficiencies. Non-limiting examples of specific conditions that can also be detected using the present invention includes Argininemia, Argininosuccinic Aciduria, Carbamoylphosphate Synthetase Deficiency 1, Citrullinemia, Homocystinuria, Hypermethioninemia, Hyperammonemia, Hyperornithinemia, Homocitrullinuria, Maple Syrup Urine Disease, Phenylketonuria (Classical Hyperphenylalaninemia/Biopterin Cofactor Deficiencies), Tyrosinemia, Methylmalonic Acidemias, Cystathionine beta-synthetase deficiency (elevated Homocysteine and methionine); Methylenetetrahydrofolate reductase deficiency (MTHFR, elevated homocysteine, low methionine).

Additional metabolites and the conditions related thereto may be found at, e.g., www.ommbid.com, which is an annotated source that provides analytes and a correlation to well-documented conditions. Relevant portions of the following references are incorporated herein by reference that teach the determination of metabolic levels of certain analytes and diseases or conditions with which they are associated: C. D. M. van Karnebeek and S. Stockler, Treatable inborn errors of metabolism causing intellectual disability: A systematic literature review, Mol. Genetics and Metabolism, 105 (2012) 368-381; Editorial, Asymmetric dimethylarginine (ADMA): Is really a biomarker for cardiovascular prognosis? Intl. Journal of Cardiology 153 (2011) 123-125; A. Meinitzer, et al., Symmetrical and Asymmetrical Dimethylarginine as Predictors for Mortality in Patients Referred for Coronary Angiography: The Ludwigshafen Risk and Cardiovascular Health Study Clinical Chemistry 57:1 (2011) 112-121; C. Wagner and M. Koury A-S-Adenosylhomocysteine—a better indicator of vascular disease than homocysteine? Am J Clin Nutr 2007; 86:1581-1585; S. Stabler, et al., Elevation of Serum Cystathionine Levels in Patients with Cobalamin and Folate Deficiency Blood Vol 81, No 12 (1993) 3404-3413; Physicians's Guide to the Laboratory Diagnosis of Metabolic Diseases, Blau, Duran and Blaskovics (Eds) (1996) Chapman and Hall, Alden Press Oxford, Chapter B, Amino Acid Analysis 24-28; and S. Stabler and R. Allen, Vitamin B12 Deficiency as a Worldwide Health Problem, Annu Rev. Nutr. (2004) 24:299-326.

The present invention can use a Liquid-Chromatography-tandem-Mass Spectrometry (LC-MS/MS) or equivalent thereof (e.g., multi-component detectors with ion drive technology), which has been introduced in clinical chemistry and is known to the skilled artisan (e.g., see Vogeser M., Clin. Chem. Lab. Med. 41 (2003) 117-126) and can include newer variants of the same with higher sensitivity. Advantages of this technology are high analytical specificity and accuracy and the flexibility in the development of reliable analytical methods. LC-MS/MS has been shown to be a robust technology, allowing its application also in a large-scale routine laboratory setting. Requirements for the preparation of sample material are limited compared to GC-MS; however, mere protein precipitation as presented by the state of the art may be sufficient for some LC-MS/MS methods, but in order to avoid ion-suppression effects for very sensitive methods, more efficient extraction methods are usually required (Annesley, T. M., Clin. Chem. 49 (2003) 1041-1044). “Off-line” or “on-line” solid phase extraction or solvent extraction are the techniques currently used to solve this problem, however, other variants can be used with the present invention.

The present invention provides methods and plasma separation devices for use in diagnostic testing using LC-MS/MS techniques. The present invention provides several advantages over the traditional blood draw method including the fact that it does not require a phlebotomist, it avoids the use of a centrifuge to separate plasma, it avoids opening of blood collection tubes and exposure to pathogens, it avoids storage of plasma in freezers and use of dry ice in transportation of specimens, and the blood collected using the present invention can be placed in a multi-barrier pouch and sealed for easy, safe storage and shipping by mail. In addition, the present invention provides an easy-to use plasma separator device that has lower costs associated with collection and transportation to allow the screening of subjects in remote areas and is a further advantage for clinical or research studies. Using LC-MS/MS techniques with this plasma separator device allows other metabolites and drugs to be tested in plasma obtained from a small drop of blood by finger stick.

The plasma separator device of the present invention offers a method for determination of plasma tHcy that includes HPLC coupled to fluorescence detection (HPLC-Flu), HPLC coupled to electrochemical detection (HPLC-EC) and LC-Mass Spectrometry (LC-MS/MS).

Several inborn errors of metabolism that lead to hyperhomocysteinemia are associated with vascular and neurological complications. Monitoring plasma total homocysteine (tHcy) during therapy is often required in the management of these cases. A simple, sensitive, and cost effective method has been validated for the analysis of tHcy using a plasma separator device (PSD) obtained from Chematics, Inc. (North Webster, Ind., US). Blood from a fingerstick is deposited on the blood introducing portion of the PSD card which contains two layers. The top layer retains blood cells while plasma diffuses to the second layer and is absorbed onto a small disc. Plasma tHcy is eluted from the disc and determined by LC-MS/MS (4000QTRAP, ABSciex). Hcy eluted at 0.9 minutes with a total analysis time of 1.5 minutes per injection. Calibration curves were shown to be linear from 2.5-80 μmol/L and the limit of quantification was 0.5 μmol/L. Intra- and inter-assay CV for plasma tHcy at three different concentrations were 8.2-8.9% and 7.7-10.7%, respectively. To validate this collection method we simultaneously collected blood from a fingerstick on the PSD and by conventional venipuncture blood draw. Samples were obtained from control subjects and patients with renal insufficiency to obtain a range of tHcy concentrations. Comparison of plasma tHcy values (PSD vs. venipuncture) demonstrated excellent correlation (r=0.96, slope=1.08; n=29; tHcy concentration range 7-36.6 μmol/L). Plasma tHcy collected on the PSD is stable for a period of 2 years when stored at 4° C.

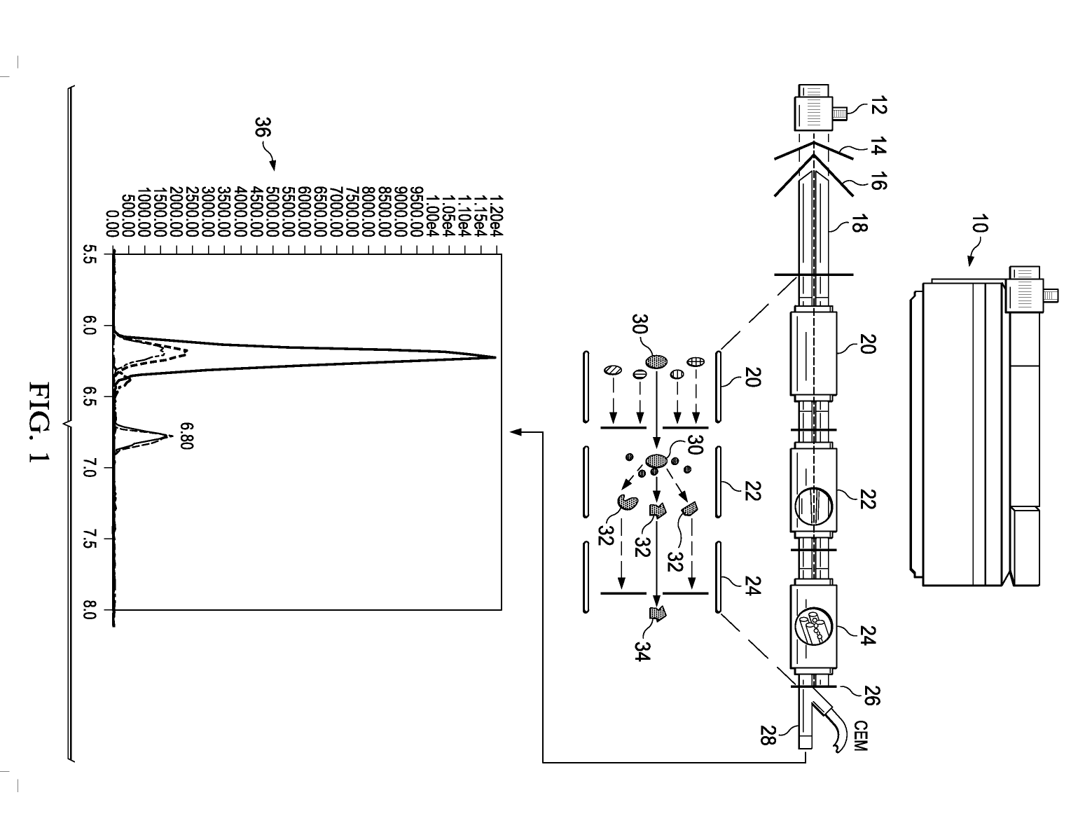

FIG. 1 is an image of a LC/MS/MS system. The LC/MS/MS system 10 includes a source 12 that is in communication with an orifice 14 and skimmer 16. The LC/MS/MS system 10 includes a high pressure cell 18 in connection with a Q1 cell 20 followed by a collision cell 22 e.g., a LINAC collision cell and a Q3 cell 24 and finally through a lens 26 and a detector 28. The Q1 cell 20 separates the sample 30, while the collision cell 20 provides a method of fragmenting the separated sample 30 into numerous fragments 32 and again separating the numerous fragments 32 in the Q3 cell 24 to a separated fragment 34 to be sent to the detector 28. The separated fragment 34 is then detected by the detector 28 and plotted 36.

The present invention provides a method and device that allows blood to be drawn at home (e.g., self administered) with no clinical visit required. The present invention provides a method and device that allows the collection times to be optimized (e.g., early morning or fasting). In addition, the present invention provides for frequent monitoring of patients with hyperhomocysteinemia, remethylation defects and CBS deficiency. The present invention provides a method and device that is particularly useful for newborns, infants and small children. In practice, the present invention reduces sample processing in clinic by eliminating centrifugation and the manual separation of plasma. The present invention provides for easy transport of samples including via direct mail with no requirement for dry ice and avoids leakage sometimes associated with transportation of regular plasma.

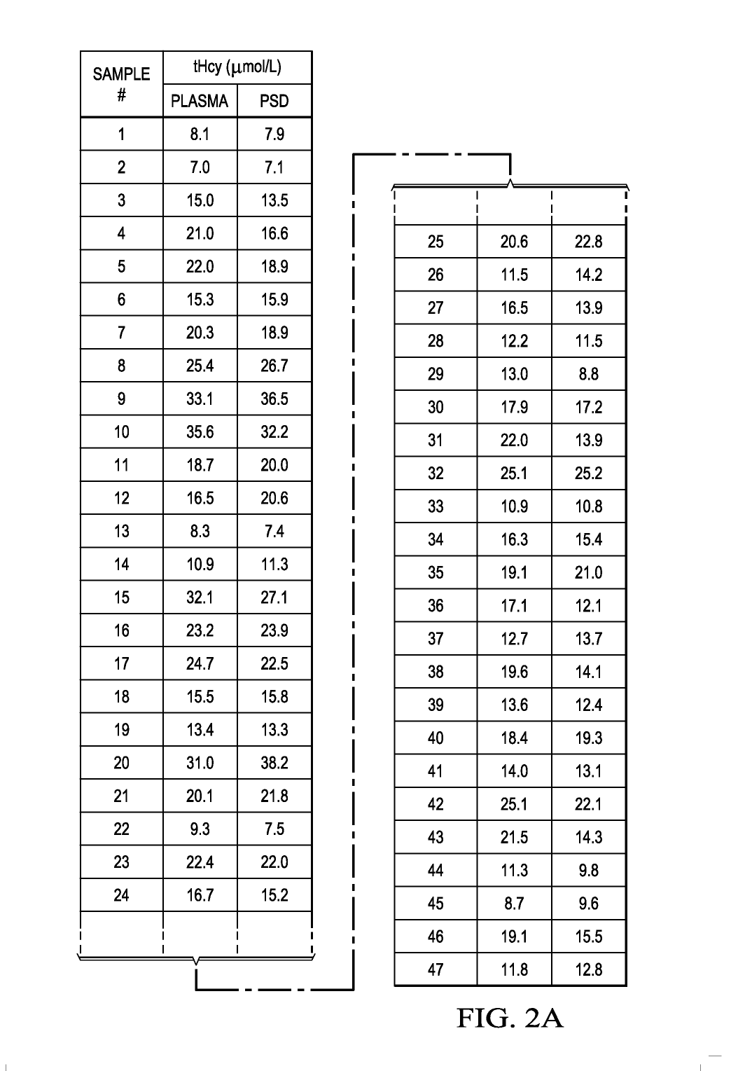

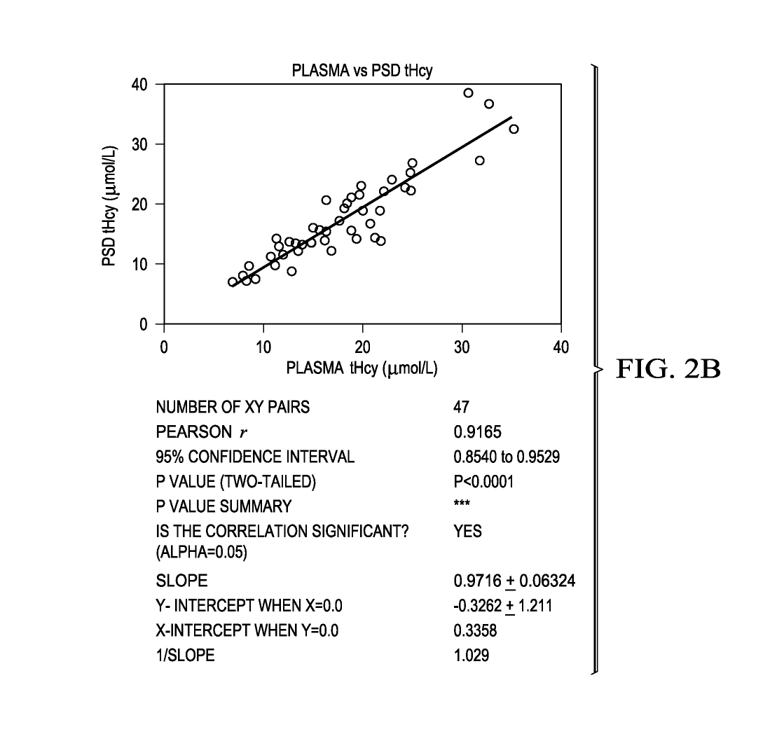

FIGS. 2A and 2B are the data and the plot that correlates tHcy testing in plasma from blood draw versus finger stick plasma separator device (PSD).

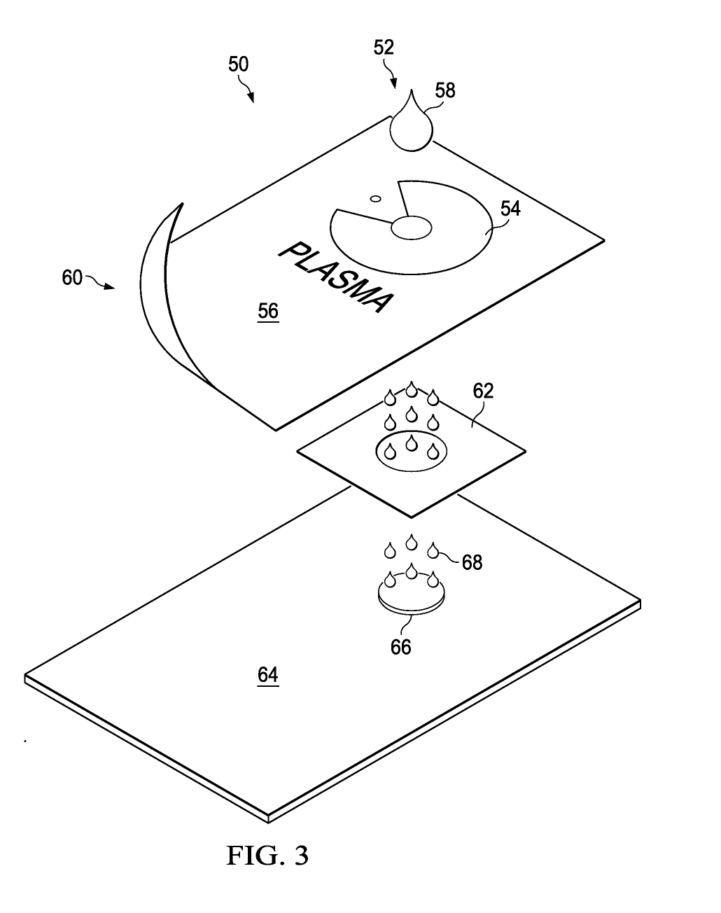

FIG. 3 is an image of a plasma separator device. The plasma separator device 50 includes a blood separator 52 with a blood introducing portion 54 on the top surface 56. A blood sample 58 may be placed on the blood introducing portion 54 and the holding member 60 removed to separate the top surface 56 from the plasma separator device 50. A semi-permeable membrane 62 is positioned between the top surface 56 and the base 64. Between the semi-permeable membrane 62 and the base 64 is a plasma collection reservoir 66 to receive the plasma 68 which can include, e.g., between about 2.00 μl and about 3.5 μl but in one embodiment the volume was about 2.4 μl (smaller and larger volumes are also encompassed, e.g., 0.1, 0.5, 1.0, 2.5, 5.0, 7.5, 10, 12.5, 15, 20, 25, 50 microliters or more).

In one embodiment, the plasma separator device 50 receives a whole blood sample on the blood introducing portion 54 from an individual. The holding member 60 removes the top surface 56 from the plasma separator device 50. The semi-permeable membrane 62 positioned between the top surface 56 and the base 64 separates out the plasma 68 that is collected in the plasma collection reservoir 66 between the semi-permeable membrane 62 and the base 64. The 2.4 μl plasma 68 sample was tested for one or more metabolites or analytes including homocysteine.

In another embodiment, the plasma separator device 50 receives a whole blood sample on the blood introducing portion 54 from an individual. The holding member 60 removes the top surface 56 from the plasma separator device 50.

The whole blood sample on the blood introducing portion 54 of the holding member 60 was processed by extraction of the DNA from blood cells and genotype analysis was preformed. The semi-permeable membrane 62 positioned between the top surface 56 and the base 64 separates out the plasma 68 that is collected in the plasma collection reservoir 66 between the semi-permeable membrane 62 and the base 64. The 2.4 μl plasma 68 sample was tested for one or more metabolites or analytes including methylmalonic acid (MMA), quantitative amino acids, and Vitamin D.

The present invention provides a procedure for determining the tHcy level using a plasma separator device 50. The plasma separator device 50 holds 2.4 μl. One sample preparation procedure for the plasma separator device 50 includes combining the plasma separator device 50, and 30 μl of 5 uM IS (d4-Hcy) containing 0.7 mg/ml Dithiothreitol (DTT) and vortex and incubate at room temp for 10 minutes. Acetonitrile 180 μl, containing 10 μl/ml formic acid is added to the sample. The sample is then vortex and centrifuged for 10 minutes at 14800 rpm at 4 C and transferred 75 μl to a LC-MS vial and injected 1 μl for analysis. Hcy and d4-Hcy were eluted isocratically on a Gemini 150×3 mm 5 μl column maintained at 32° C. with a mobile phase consisting of 75% acetonitrile and 0.1% formic acid. Both Hcy and d3-Hcy eluted at 0.9 minutes with a total analysis time of 1.5 minutes per sample.

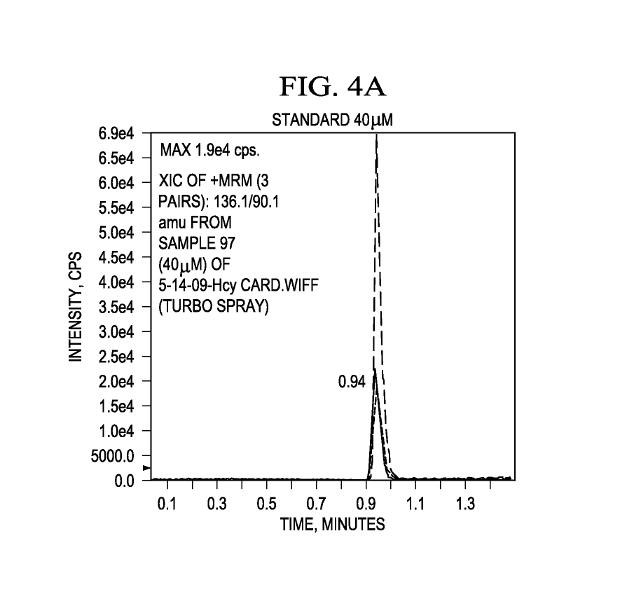

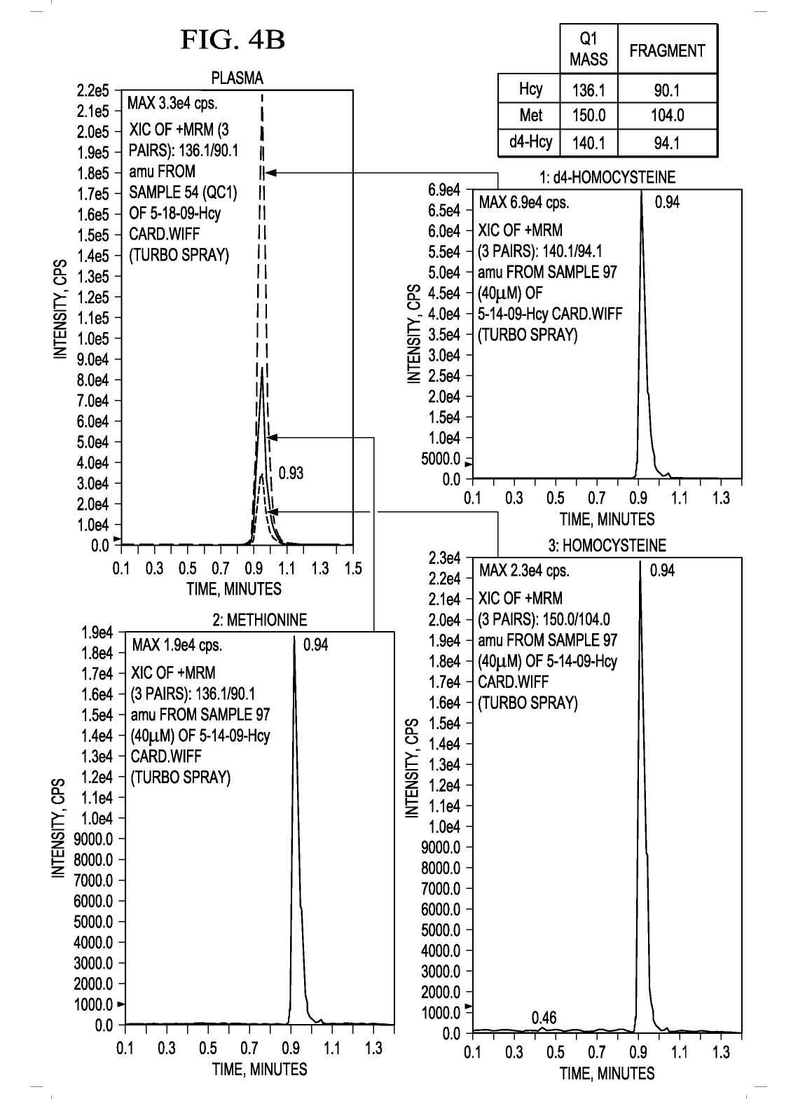

FIGS. 4A and 4B are images of the plot of the LC-MS/MS (MRM) determination of Plasma tHcy for a standard (FIG. 4A) and the sample (FIG. 4B). The plot clearly shows the d4-Hcy (1), Methionine (2), and Hcy (3) peaks.

| | Q1 Mass | Fragment |
|---|---|---|
| Hcy | 136.1 | 90.1 |
| Met | 150.0 | 104.0 |
| d4-Hcy | 140.1 | 94.1 |

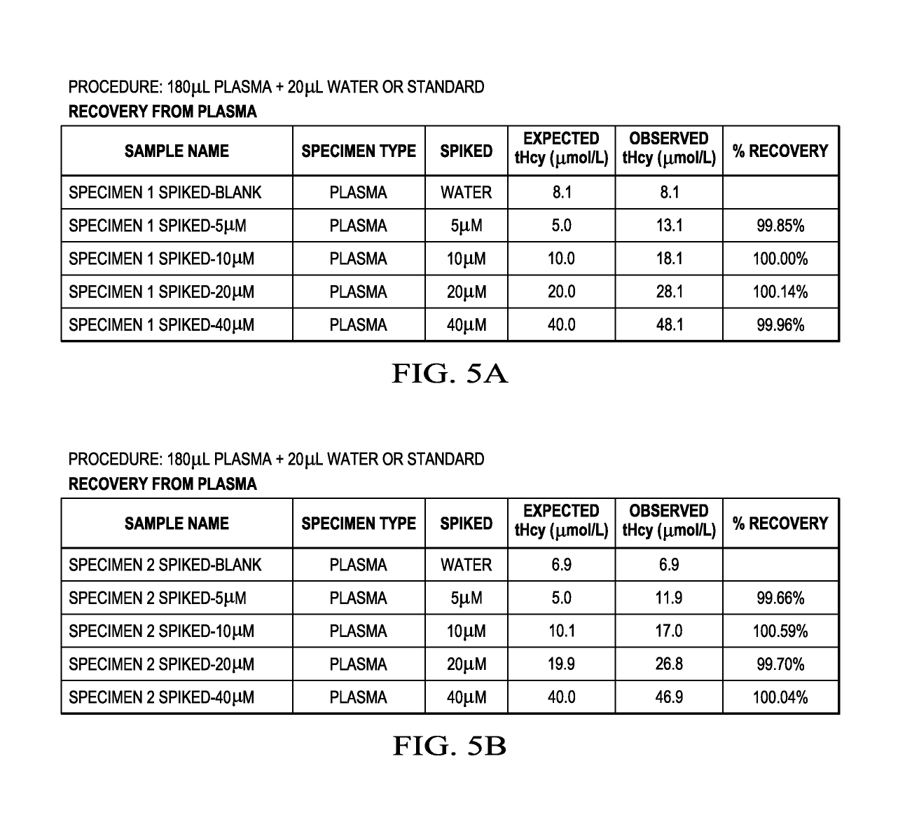

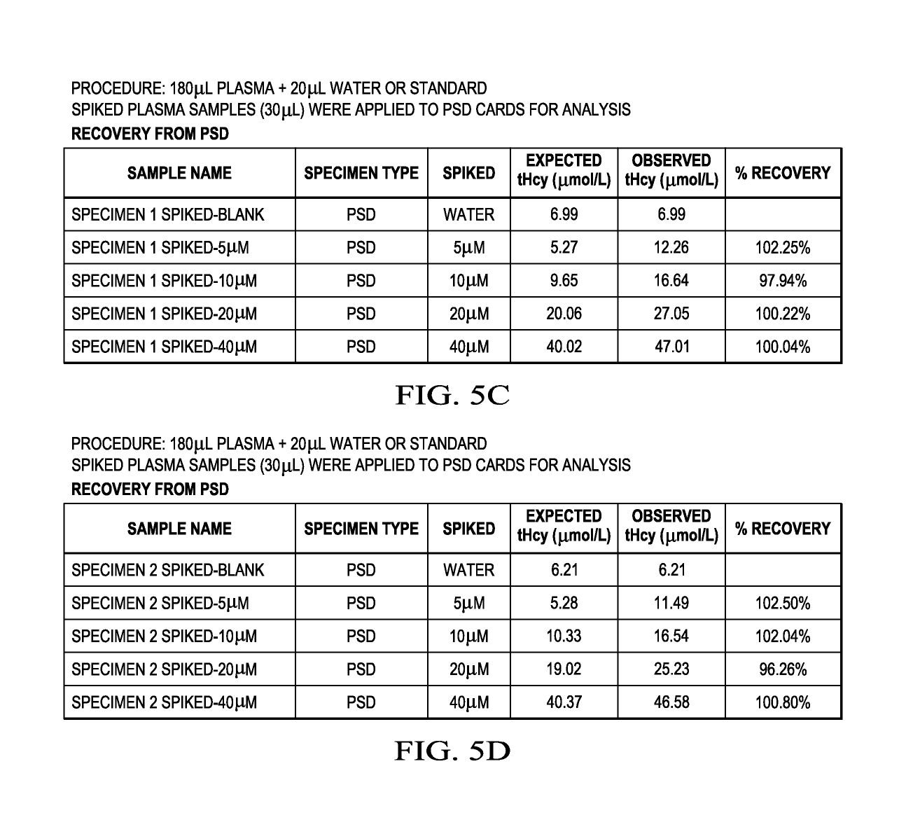

FIGS. 5A-5D are tables demonstrating recovery from a plasma sample and a PSD sample for subject 1 and 2 and includes expected and observed concentrations for tHcy and amount of spiked standard recovered.

In one sample preparation method, a single 3/16-inch dried blood spot punch is extracted with an acidified acetonitrile and water solution containing dithiothreitol (DTT), d3-methylmalonic acid (d3-MMA), d3-methylcitric acid (d3-MCA), and d8-homocysteine. During 1 hour of agitation, free homocysteine, protein-bound homocysteine, and the added d8-homocysteine internal standard are reduced to homocysteine. The extract is transferred and then evaporated under heated nitrogen. Dried residue is treated with 3 N HCl in n-butanol to form butylesters. After evaporation of the butanol, the residue is reconstituted, centrifuged, and the supernatant is transferred to microvials and subjected to LC-MS/MS analysis.

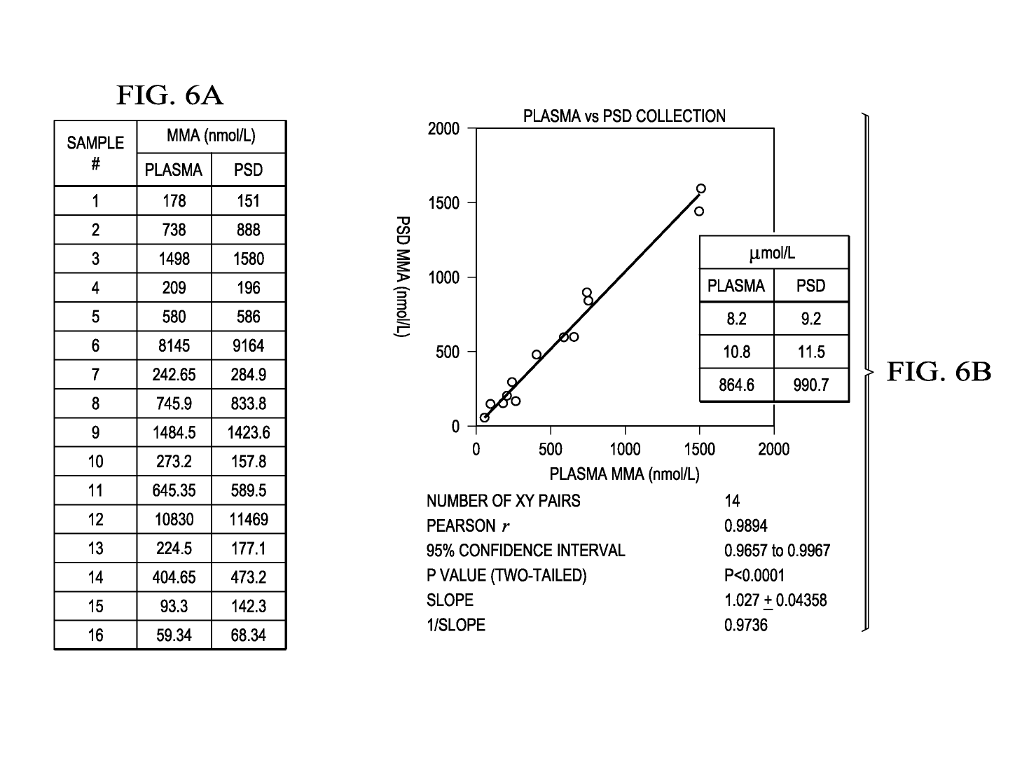

The present invention provides a procedure for determining the MMA level using a plasma separator device 50. The plasma separator device 50 holds 2.4 ul. One sample preparation procedure for the plasma separator device 50 includes combining the plasma separator device 50, and 80 ul of 5 uM IS (d3-MMA) containing and vortex and incubate at room temp for 10 minutes. 70 μl of sample solution is loaded into Amicon Ultra 0.5 mL 10,000 MW cutoff ultra-centrifugation filter and centrifuge at 14800 rpm for 10 minutes at room temperature. The filtrate is removed and loaded into an MTP with 10 μl injection for analysis. MMA and d3-MMA were eluted isocratically on a Waters Symmetry 100×2.1 mm 3.5μ column maintained at 32° C. with a mobile phase consisting of 10% acetonitrile and 0.1% formic acid. Both MMA and d3-MMA eluted at 1.2 minutes with a total analysis time of 2 minutes per sample. FIGS. 6A-6B are the data and the plot that correlate MMA testing in plasma from plasma versus plasma spotted on PSD.

The table below compares the analyte detected for association with a disease. For example, the present invention provides for the detection of numerous analytes through a single PSD sample to identify and diagnose a disorder, e.g., nutritional deficiencies, vascular risk factors, inborn errors of metabolism, amino acidopathies, renal insufficiency and so on. Other compounds may also be analysed using the present invention, e.g., glutathione.

List of metabolites and associated disorders that can
be determined using the Plasma Separation Device (PSD).

| | LC-MS/MS | | | | | | |
|---|---|---|---|---|---|---|---|
| | Method | | | | | | Δ |
| | 1 | 2 | 3 | 4 | 5 | Disorder | Value |
| total Homocysteine (tHcy) | X | | | | | A, B, C | ↑ |
| Methylmalonic acid (MMA) | | X | | | | A | ↑ |
| Methionine | X | | X | | | C | ↑↓* |
| S-adenosylmethionine (SAM) | | | X | | | C | ↑↓* |
| S-adenosylhomocysteine | | | X | | | B, C, D | ↑ |
| (SAH) | | | | | | | |
| Betaine | | | X | | | A | ↓ |
| Choline | | | X | | | A | ↓ |
| Asymmetric dimethylarginine | | | X | | | B, D | ↑ |
| (ADMA) | | | | | | | |
| Symmetric dimethylarginine | | | X | | | B, D | ↑ |
| (SDMA) | | | | | | | |
| creatinine | X | | | | | D | ↑↓* |
| Amino Acids (full screen | | | | X | | C | ↓ |
| 42 compounds) | | | | | | | |
| Vitamin D | | | | | X | A | ↓ |
| Vitamin B1 (thiamine) | | | | | X | A | ↓ |
| Vitamin B2 (Riboflavin) | | | | | X | A | ↓ |
| Vitamin B3 (Niacin) | | | | | X | A | ↓ |
| Vitamin B4 (adenine) | | | | | X | A | ↓ |
| Vitamin B5 (pantothenic acid) | | | | | X | A | ↓ |
| Vitamin B6 (Pyridoxine) | | | | | X | A | ↓ |
| Vitamin B7 (Biotin) | | | | | X | A | ↓ |

Key - Disorder
A Nutritional deficiencies
B Vascular risk factor
C In-born errors of Metabolism—Amino Acidopathies
D Renal insufficiency
↑↓* may find increased or decreased level depending on metabolic defect

Some of the numerous analytes that can be detected using the PSD of the present invention include Homocysteine (tHcy, Methylmalonic acid (MMA), Methionine, S-adenosylmethionine (SAM), S-adenosylhomocysteine (SAH), Betaine, Choline, Asymmetric dimethylarginine (ADMA), Symmetric dimethylarginine (SDMA), creatinine, Amino Acids (full screen 42 compounds), glutathione, Vitamin D, Vitamin B1 (thiamine), Vitamin B2 (riboflavin), Vitamin B3 (niacin), Vitamin B4 (adenine), Vitamin B5 (pantothenic acid), Vitamin B6 (pyridoxine), and Vitamin B7 (biotin). The detection of one or all of the analytes is contemplated herein.

Measurement of S-Adenosylmethionine, S-Adenosylhomocysteine, ADMA, and SDMA by LC/MS/MS in Plasma Separator Device. Method 1-3 in the above table employ stable-isotope dilution liquid chromatography-electrospray injection tandem mass spectrometry (LC-ESI-MS/MS) to determine SAM, SAH, ADMA, SDMA, methionine, choline, betaine, and cystathionine in plasma or serum. Calibrators and internal standards (2H3-SAMe, L-(2,3,3,4,4,5,5-2H7-ADMA, 2H3-methionine, 2H3-choline, 2H3-betaine, 2H3-cystathionine) were included in each analytical run for calibration. 1 mM stock solutions of each standard were diluted in distilled water to perform a 5-point calibration curve over the following concentration ranges of 12.5-400 nmol/L (SAM and SAH), 125-2000 nmol/L (ADMA, SDMA, and cystathionine), and 5-80 μmol/L (methionine, choline, and betaine).

Sample preparation utilized microcentrifugal filter units, Microcon YM-10, 10 kDa NMWL (Millipore, USA). Samples were prepared by the addition of 100 μL mobile phase A containing 10-50 μmol/L labeled-isotope internal standards to a single PSD or 2.4 μl standard followed by vortex and incubation at room temperature for 10 minutes. 90 μl of incubation solution was added to a microcentrifugal filter unit and centrifuged for 20 min at 14800×g at 4° C. Sample filtrate was removed and transferred to a microtiter plate for analysis. 10 μl was injected into the LC-MS system, a Shimadzu Prominence LC System interfaced with a 4000 QTRAP® LC-MS/MS (Applied Biosystems).

Chromatographic separation was achieved on a 250×2.0 mm EZ-faast analytical column (Phenomenex) maintained at 33° C. at a flow of 250 μL/min with a bianary gradient with a total run time of 12 minutes. Solvents for HPLC were: (A) 4 mM ammonium acetate, 0.1% formic acid, 0.1% heptafluorobutyric acid (pH=2.5); (B) 100% methanol and 0.1% formic acid. The initial gradient condition was 75% A: 25% B and was increased in a linear fashion to 100% B in 6 min and held constant for 1 min. At 7.1 min the mobile phase was reset to initial conditions for 5 minutes. The flow from the column was delivered to the ESI source from the period of 3 to 8 min, otherwise the flow was diverted to waste. The compounds were detected by MRM using positive ESI with a dwell time of 30 ms. The curtain gas was set at 15 L/min, and source gas 1 and 2 were set at 60 L/min. The heater was set to 700° C. with an ionspray voltage of 5000V and CAD gas (nitrogen) was set at 3.5×10e-5 Ton. Analyte specific MRM transitions monitored, declustering potentials (DP), entrance potential (EP), collision energy (CE), and collision exit potential (CXP) are shown in the previous table. All data were collected using Analyst software version 1.4.2.

SAM, SAH, ADMA, SDMA, methionine, cystathionine, choline and betaine were resolved by a gradient to 100% methanol with retention times of 7, 6.6, 6.5, 6.5, 4.3, 6.1, and 3.8 minutes, respectively. HPLC chromatographic conditions did not produce complete separation of ADMA and SDMA, but they were completely discerned by their different fragmentation pattern in the spectrometer mass working in the MS-MS mode. The observed m/z values of the fragment ions were m/z 399→250 for SAM, m/z 385→136 for SAH, m/z 402→250 for 2H3-SAM, m/z 203→46 for ADMA, m/z 203→172 for SDMA, m/z 210→46 for 2H7-ADMA, m/z 150→104 for methionine, m/z 153→107 for 2H3-methionine, m/z 223→134 for cystathionine, m/z 227→138 for 2H4-cystathionine, m/z 104→45 for choline, m/z 108→49 for 2H4-choline, m/z 118→59 for betaine, and m/z 121→61 for 2H3-betaine.

Measurement of Vitamin B species and vitamin D by LC/MS/MS in Plasma Separator Device. Method 5 has been modified to accommodate the small volume size associated with the PSD. In brief, 30 μl solution containing stable isotopes of the B vitamins was added to the PSD or 2.4 μl standard and incubated on ice in the dark, protected from light for 10 minutes. Next, 30 μl 6% TCA solution containing stable isotopes of the B vitamins is added to deproteinize the sample. The sample is vortexed and incubated on ice, protected from light for 1 hour. Following incubation, the sample is centrifuged at 14800 rpm at 4° C. for 10 minutes. Supernatant is loaded in microtiter plate and 5 μl is injected onto LC-MS/MS system. Separation of B vitamins is achieved by a gradient on a Agilent Eclipse Plus C18 150×3 mm 3.5 μl.

Quantitative Plasma Amino Acid Screen by LC/MS/MS in Plasma Separator Device. Method 4 includes a quantitative amino acid screen from a PSD will be performed by modifying the sample preparation of the aTRAQ method offered by AB Sciex to accommodate the small volume size associated with the PSD. In use a subject may take a blood sample and place it on the PSD. The PSD sample is then analyzed to determine Homocysteine (tHcy), Methylmalonic acid (MMA), Methionine, S-adenosylmethionine (SAM), S-adenosylhomocysteine (SAH), Betaine, Choline, Asymmetric dimethylarginine (ADMA), Symmetric dimethylarginine (SDMA), creatinine, Amino Acids (full screen 42 compounds), Vitamin D, Vitamin B1 (thiamine), Vitamin B2 (riboflavin), Vitamin B3 (niacin), Vitamin B4 (adenine), Vitamin B5 (pantothenic acid), Vitamin B6 (pyridoxine), and Vitamin B7 (biotin). The results can then be used to aid in the diagnosis of nutritional deficiencies, vascular risk factor, and in-born errors of metabolism.

The Plasma Separator Device (PSD) has a blood separation member and the blood separation member is covered and held by a holding member. The holding member includes a base film in the lower side and a covering film in the upper side, the blood separation member is fixedly sandwiched between the base film and covering film. When fixedly covering and holding the blood separation member with the holding member, by adhering them without leaving any clearance between, it is possible to isolate high purity plasma or serum from whole blood. A blood introducing portion is formed on the upper surface of a proximal end portion of the covering film and a plasma or serum sampling aperture is perforated in a distal end portion of the holding member on the opposite side thereof of the blood introducing portion.

The blood separation member exposes the outside at the blood introducing portion and is covered with a protection member for protection from damage or the like. Any material may be used as the protection member as far as it does not become spherical by the action of surface tension as a result of blood permeation, and a plastic material such as nylon can be used.

The shape of the blood introducing portion is not specifically limited and may be either a circle or any other shape such as polygons. The shape of the blood introducing portion can also be formed in such a way that the proximal end portion of the covering film is all peeled off to form a large opening portion. Although the blood introducing portion is preferably covered with the protection member, it is needless to say that the function and result of the present invention can be achieved even in an embodiment where the blood separation member is exposed to the outside air without providing the protection member.

The sampling aperture may be perforated on the upper surface of the holding member, on the bottom surface thereof, or on any portion of side surfaces of the distal end portion without any specific limitation. The size of the plasma or serum sampling aperture may be a circle or square of from 0.02 mm to 1 mm.

The fibrous materials and/or porous materials used as the blood separation member can include inorganic fibers, e.g., glass fibers and asbestos; natural organic fibers, e.g., cotton, pulp, silk and the like; semi-synthetic fibers or synthetic fibers e.g., cellulose, cellulose acetate, polyester, polypropylene, polyurethane, polyamide, polyvinyl formal, polyethylene, polyvinyl chloride, viscose rayon and the like.

The blood separation member may be fibrous materials and/or porous materials coated with coating materials e.g., hexylene glycol, propanol with a butoxy group and acrylamide with a butoxy group. The coating materials may be used either alone or jointly consisting of two or more kinds e.g., glass fiber filter, glass fibers coated with one or more kinds selected from the group consisting of hexylene glycol, propanol with a butoxy group and acrylamide with a butoxy group.

A size of the blood separation member is required to be at least of a volume corresponding to a blood sample amount. A shape thereof is not specifically limited and may be any selected shape from the group consisting of a quadrangle, a triangle, other polygons, a circle, an ellipse, a shape of a tapered battledore plate with a narrower distal end and the like.

The thickness of the blood separation member is such that it allows the separation of a blood cell portion from a plasma or serum portion in whole blood, the blood cell portion remains in the blood separation member in whole blood supplied from the blood introducing portion, and the plasma or serum portion is caused to migrate in a traverse direction, that is in a direction toward the plasma or serum sampling aperture, and hence the thickness of the blood separation member is set such that the blood separation member is filled with the blood cell portion from the upper surface to the bottom surface thereof and plasma or serum flows in the traverse direction in the blood cell separation member. The size of the blood separation member has only to be properly determined based on an amount of plasma or serum necessary for an examination without a specific limitation thereon.

The plasma or serum separating method of the present invention employs the plasma separator device and thereby, plasma or serum can be efficiently isolated from even a small amount of blood without leaking blood cell components or causing hemolysis.

In the first aspect the plasma sampling method according to the present invention, includes a lancing device that is stuck in a blood sampling portion to cause the portion to bleed. The blood sampling portion is not specifically limited and for example, a hand, a foot or the like. After the bleeding, the blood introducing portion or the protection member of the plasma separator device is brought into contact with the bleeding site to sample blood and to supply the blood through the blood introducing portion.

The absorbed blood migrates in the blood separation member from the blood introducing portion to the sampling aperture at the distal end portion and a difference in migrating speed between plasma or serum and red blood cells is used so that red blood cells are isolated in the blood introducing portion side while plasma or serum is isolated in the plasma or serum sampling aperture side; thereby plasma or serum being isolated in the blood separation member. With the holding member, especially the covering film being transparent or semi-transparent, the separation process can be visually confirmed through the covering film. A sampling amount of plasma or serum is determined by a hematocrit value of the blood and a plasma or serum separation ability of the blood separation member. The isolated sample circle can be taken out from the plasma separator device for analyzing.

With the plasma separator device and method of the present invention, high purity plasma or serum can be easily obtained from a small amount of blood without the use of a centrifuge. The present invention provides a plasma or serum sample that can be directly subjected to quantitative analysis.

The blood separation laminate is composed of a first layer made of a blood separation member, a second layer made of a hemolysis blocking member and a third layer made of a plasma or serum absorbing member. The first layer exerts blood separation action and is made of a blood separation member. The second layer exerts blocking action so that hemolysis does not spread to the third layer and is made of porous film materials such as nitrocellulose and cyclopore. The third layer exerts absorbing action of the isolated plasma or serum and is made of water-absorbing materials such as glass fibers, cellulose, non-woven fabrics, filter paper or the like.

The present invention provides for the reduction in the volume of blood collected in pre-clinical studies, which has a significant impact on animal studies and data quality. For example, the number of rodents needed for a study can be reduced by up to 75%, and the quantity of compound needed for testing can also be greatly reduced. This is particularly applicable in instances where a composition has not yet been optimized and is costly, time-consuming, and difficult to achieve and purify. The present invention provides a method for producing high quality data in pre-clinical studies by increasing the number of time points that can be added without the need for additional rodents and allowing for serial pharmacokinetic (PK) profiling. Serial PK profiling eliminates the variability between animals observed when using composite profiling and greatly improves the quality of the data.

The present invention provides for drug development programs by providing methods using less invasive blood sampling, which is especially beneficial for pediatric studies and in critically ill patients. In addition, the present invention allows shipping, handling, and storage of samples at room temperature and under normal conditions without the need for specialized biohazard precautions, since pathogens like HIV and hepatitis B are inactivated. The present invention reduces the need for specialized equipment at clinical sites (e.g., refrigerated centrifuge, monitored freezers, etc.) and allows clinical studies in emerging countries.

Analysis of Total Homocysteine and Methylmalonic acid in plasma by LC-MS/MS from a fingerstick.

The present invention was used to determine that several inborn errors of metabolism can lead to moderate and severe hyperhomocysteinemia that is associated with vascular and neurological complications. Monitoring plasma total homocysteine (tHcy) during therapy is often required in the management of these cases. The simple, sensitive and cost effective LC-MS/MS method of the present invention was developed for the analysis of tHcy obtained using a plasma separator device (PSD). This device was also used and validated for the determination of methylmalonic acid (MMA), a marker of B12 deficiency.

Standards for Hcy and MMA were obtained from Sigma and isotope-labeled standards 2H4-Hcy (Cambridge Isotopes) and 2H3-MMA (CDN Isotopes). MMA calibrators and quality control material were obtained from Recipe Chemicals (Germany).

A drop of blood from a fingerstick is deposited on the test area of the CHEMCARD™ (Chematics, North Webster, USA) (FIG. 1). The plasma is separated from the rest of the blood sample by filtration and absorption within three minutes, leaving a single disc containing 2.4 μl plasma. The card with attached plasma disc is placed in a multi-barrier pouch for shipping and storage until time of analysis. Extraction of plasma tHcy and MMA is performed by incubating the plasma disk at room temperature for 10 minutes with dithiothreitol and internal standards (2H4-Hcy and 2H3-MMA) (shown in FIG. 3). Plasma and PSD levels of tHcy and MMA were measured by a modification of the stable-isotope dilution liquid chromatography-electrospray injection tandem mass spectrometry (LC-ESI-MS/MS) previously described (Ducros V, Belva-Besnet H, Casetta B, Favier A. A robust liquid chromatography tandem mass spectrometry method for total plasma homocysteine determination in clinical practice. Clin Chem Lab Med 2006; 44(8):987-990).

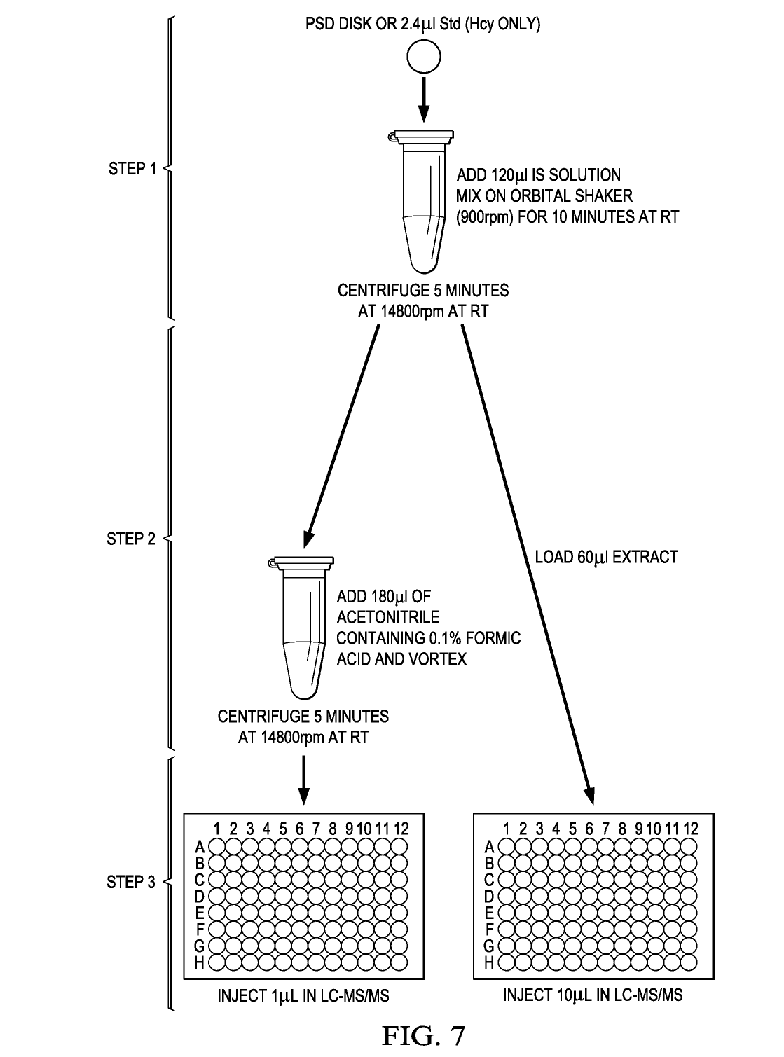

FIG. 7 shows one of the procedures for sample extraction and analysis of the present invention. The PSD disk or 2.4 microliters of the standard (Hcy only) are placed in a tube with 10 microliters of IS solution and mixed in an orbital shaker at 900 rpm for 10 minutes at room temperature. The tube is centrifuged for 5 minutes at 14,800 rpm at room temperature. The liquid phase is separated into two samples. For the first sample, 60 microliters of the liquid phase are separated and 10 microliters are loaded into an LC-MS/MS to measure the MMA. To the remainder, 180 microliters of Acetonitrile containing 0.1% formic acid is added and vortexed. The tube is centrifuged and 1 microliter is injected into an LC-MS/MS to measure the tHcy.

The following is a summary of the tHcy analytical method. Instrumentation: Shimadzu Prominence HPLC coupled to an ABSciex 4000 QTRAP; HPLC Column: Gemini 150×3 mm 5μ (Phenomenex): HPLC Eluent (Isocratic): 0.1% formic acid in 75% Acetonitrile (flow=0.6 ml/min). Retention times: Hcy and 2H4-Hcy4=0.9 min. Calibration curve range 2.5-80 μmol/L (2.4 μl prepared in water). Sample Preparation: See FIG. 7. Internal standard (IS) solution: 10 μM 2H4-Hcy and 2 μM 2H3-MMA prepared in water containing 0.1% DTT. The following table summarizes the MS/MS settings:

MRM transition

| | | Q1 | Q3 | DP | CE | CXP |
|---|---|---|---|---|---|---|
| Analyte | Ion Mode | (m/z) | (m/z) | (V) | (V) | (V) |
| Hcy | Positive | 136.1 | 90.1 | 31 | 17 | 6 |
| ²H₄-Hcy | Positive | 140.1 | 94.1 | 31 | 17 | 6 |

The following is a summary of the MMA analytical method. Instrumentation: Shimadzu Nexera HPLC coupled to an ABSciex 5500 QTRAP HPLC Column Synergi Hydro-RP 250×3 mm 4μ (Phenomenex). HPLC Eluents A—0.1% formic acid in water B—0.1% formic acid in methanol. The following table includes the gradient profiles:

| | |
|---|---|
| Gradient profile | Diverter Valve |

| TIME | A % | B % | Time | Position |
|---|---|---|---|---|
| Initial | 100 | 0 | Initial | Offline |
| 4.0 | 45 | 55 | 4.5 | Online |
| 4.1 | 100 | 0 | 6.0 | Offline |
| 7.0 | End | | | |

Retention times MMA and 2H3-MMA=5.5 min Calibration curve range 221-1499 nmol/L (Recipe). The sample preparation followed FIG. 7. Internal standard (IS) solution: 10 μM 2H4-Hcy and 2 μM 2H3-MMA prepared in water containing 0.1% DTT. The following table include the MS/MS Settings:

MRM transition

| | | Q1 | Q3 | DP | CE | CXP |
|---|---|---|---|---|---|---|
| Analyte | Ion Mode | (m/z) | (m/z) | (V) | (V) | (V) |
| MMA | Negative | 117.1 | 73.1 | −4D | −13 | −3 |
| ²H₃-MMA | Negative | 120.1 | 76.1 | −4D | −13 | −3 |

Using the present invention, it was possible to consistently measure tHcy and MMA within and across assay. For example, the following table demonstrates the precision of the method both intra- and inter-assay.

| | Intra-assay | | Inter-assay | | | |
|---|---|---|---|---|---|---|
| | Precision | | Precision | | | |
| | Level 1 | Level 2 | Level 1 | Level 2 | Linearity | LOQ |
| Analyte | (CV %) | (CV %) | (CV %) | (CV %) | (μM) | (μM) |
| tHcy (μM) | 6.1 ± 0.5 | 60.4 ± 4.9 | 12.2 ± 1.3 | 27.4 ± 2.1 | 1-160 | 1 |
| n = 20 | (8.9%) | (8.2%) | (10.7%) | (7.7%) | | |
| MMA (nM) | 341 ± 46.8 | 631 ± 48.2 | 273 ± 41.7 | 603 ± 66.1 | 0.2-80 | 0.2 |
| n = 12 | (13.7%) | (7.7%) | (15.3%) | (10.9%) | | |

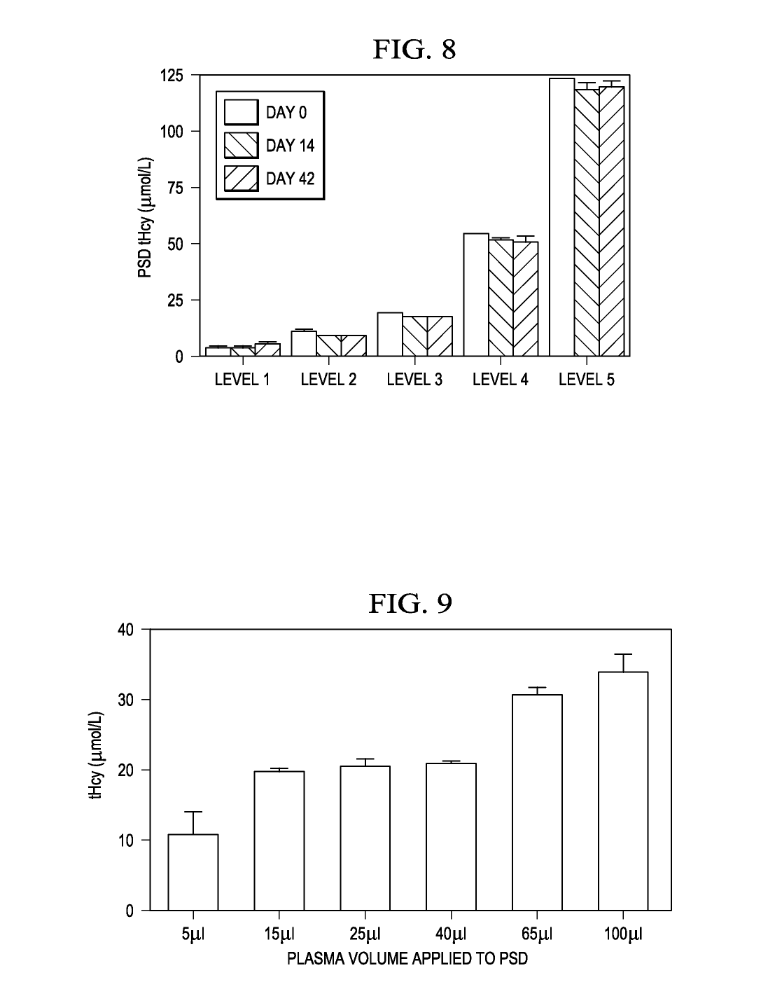

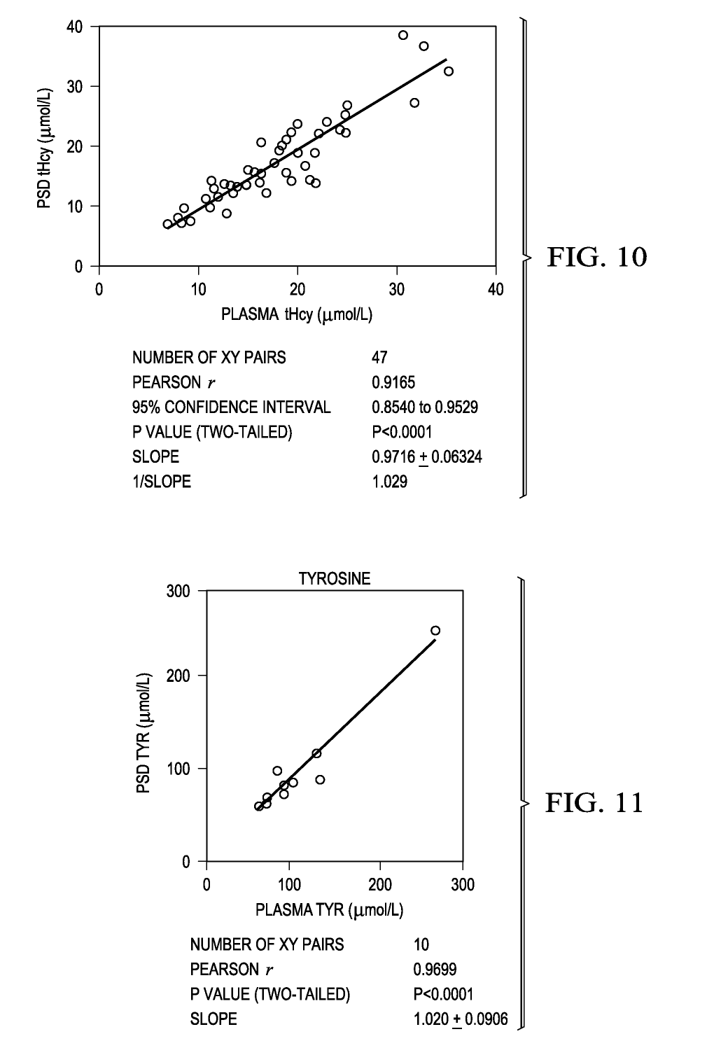

FIG. 8 is a graph that shows the stability of tHcy on PSD at room temperature that compares day 0, day 14 and day 42. FIG. 9 is a graph that shows the volume-dependence for PSD tHcy with varying amounts of plasma applied to the PSD. FIG. 10 is a graph that shows the results from simultaneous venipuncture and PSD collection in subjects with ESRD.

It was found that the PSD of the present invention offers several advantages over the traditional blood draw method: 1. Does not require a phlebotomist; 2. Avoids the use of a centrifuge to separate plasma; 3. Avoids opening of blood collection tubes and exposure to pathogens; 4. Avoids use of dry ice in transportation of specimens; 5. Reduced storage space of plasma in freezers; and 6. Blood collected using the PSD can be placed in a multi-barrier pouch and sealed for easy, safe storage and shipping by mail. Sample handling, shipping, and storage requirements collectively reduce the costs associated with plasma tHcy and MMA testing.

Additionally, preliminary studies in progress suggest other metabolites (i.e. S-adenosylmethionine, S-adenosylhomocysteine, methionine, asymmetric dimethylarginine, symmetric dimethylarginine and other amino acids) may also be determined using this technique. This mode of collection is useful for monitoring therapy in cases of hyperhomocysteinemia, but may also be useful for screening subjects in remote areas for clinical or research studies.

PKU Monitoring From a fingerstick using a plasma separator device.

The present invention was also used in a new method of collecting plasma from a finger stick that can be used as an improved home based collection method for phenylalanine analysis.

Briefly, the present invention was used to analyze phenylalanine. The management of phenylketonuria (PKU) involves both dietary restriction of phenylalanine and frequent monitoring of an individual's blood level of phenylalanine. The present inventors have developed and validated a simple, accurate, and cost effective method for the analysis of phenylalanine and tyrosine using a plasma separator device (PSD).

Methods: Fingerstick blood (1 or 2 drops) is deposited on the PSD card, where the top layer retains cells while plasma filters through to a disc on the second layer. Plasma is extracted (2.4 μl) from the disc, and phenylalanine and tyrosine determined by liquid chromatography-electrospray tandem mass spectrometry (4000QTRAP, ABSciex).

Results: The method allows for accurate determination of both phenylalanine and tyrosine over a wide linear working range (10-2000 μmol/L) with total analytical imprecision less than 10%. Comparison of plasma phenylalanine and tyrosine values (PSD vs plasma) demonstrated excellent correlation (Pearson r=0.992, slope=1.1; Pearson r=0.969 slope=1.02; n=10, respectively). Plasma phenylalanine and tyrosine collected on the PSD is stable for a period of 2 years when stored at 4° C.

The method allows for the accurate determination of Tyrosine and Phenylalanine from a PSD, The following Tables demonstrate the results of using the PSD of the present invention:

Tyrosine

| | | Expected | Observed | |
|---|---|---|---|---|
| | Recovery | Value | Value | |
| Sample # | protocol | (μmol/L) | (μmol/L) | % Recovery |
| 1 | Water Spiked | NA | 79 | NA |
| 2 | 500 μM Spiked | 579 | 591 | 102.1% |
| 3 | 1000 μM Spiked | 1079 | 1070 | 99.2% |
| 4 | 2000 μM Spiked | 2079 | 2075 | 99.8% |

Phenylalanine

| | | Expected | Observed | |
|---|---|---|---|---|
| | Recovery | Value | Value | |
| Sample # | protocol | (μmol/L) | (μmol/L) | % Recovery |
| 1 | Water Spiked | NA | 138 | NA |
| 2 | 500 μM Spiked | 638 | 644 | 100.9% |
| 3 | 1000 μM Spiked | 1138 | 1120 | 98.4% |
| 4 | 2000 μM Spiked | 2138 | 2150 | 100.6% |

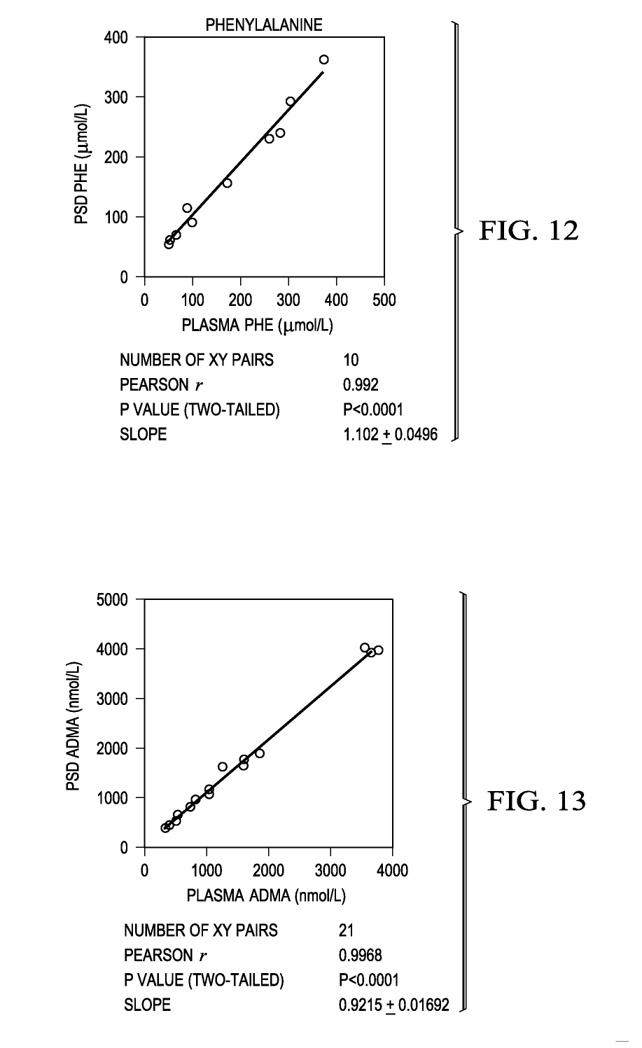

FIG. 11 is a graph that shows the correlation for Tyrosine in plasma versus PSD. FIG. 12 is a graph that shows the correlation for Phenylalanine in plasma versus PSD.

The present invention was also used to measure ADMA, SDMA and Arginine recovery. The following tables show the results for ADMA, SDMA and Arginine recovery using the present invention.

| | | | Expected | Observed | |
|---|---|---|---|---|---|
| | | ADMA | Value | Value | % |
| Sample # | Sample ID | Recovery protocol | (nmol/L) | (nmol/L) | Recovery |
| 1 | PSD A | Water Spiked | | 510 | |
| 2 | PSD A | 500 nM ADMA/SDMA + 25 μM Arg | 1010 | 1070 | 105.9% |
| 3 | PSD A | 1000 nM ADMA/SDMA + 100 μM Arg | 1510 | 1630 | 107.9% |
| 4 | PSD A | 3000 nM ADMA/SDMA + 300 μM Arg | 3510 | 3990 | 113.7% |
| 5 | PSD B | Water Spiked | | 640 | |
| 6 | PSD B | 500 nM ADMA/SDMA + 25 μM Arg | 1140 | 1140 | 100.0% |
| 7 | PSD B | 1000 nM ADMA/SDMA + 100 μM Arg | 1640 | 1740 | 106.1% |
| 8 | PSD B | 3000 nM ADMA/SDMA + 300 μM Arg | 3640 | 3890 | 106.9% |
| 9 | PSD C | Water Spiked | | 821 | |
| 10 | PSD C | 500 nM ADMA/SDMA + 25 μM Arg | 1321 | 1600 | 121.1% |
| 11 | PSD C | 1000 nM ADMA/SDMA + 100 μM Arg | 1821 | 1860 | 102.1% |
| 12 | PSD C | 3000 nM ADMA/SDMA + 300 μM Arg | 3821 | 3910 | 102.3% |

| | | | Expected | Observed | |
|---|---|---|---|---|---|
| | | SDMA | Value | Value | % |
| Sample # | Sample ID | Recovery protocol | (nmol/L) | (nmol/L) | Recovery |
| 1 | PSD A | Water Spiked | | 359 | |
| 2 | PSD A | 500 nM ADMA/SDMA + 25 μM Arg | 859 | 913 | 106.3% |
| 3 | PSD A | 1000 nM ADMA/SDMA + 100 μM Arg | 1359 | 1390 | 102.3% |
| 4 | PSD A | 3000 nM ADMA/SDMA + 300 μM Arg | 3359 | 3690 | 109.9% |
| 5 | PSD B | Water Spiked | | 701 | |
| 6 | PSD B | 500 nM ADMA/SDMA + 25 μM Arg | 1201 | 1170 | 97.4% |
| 7 | PSD B | 1000 nM ADMA/SDMA + 100 μM Arg | 1701 | 1780 | 104.6% |
| 8 | PSD B | 3000 nM ADMA/SDMA + 300 μM Arg | 3701 | 3870 | 104.6% |
| 9 | PSD C | Water Spiked | | 554 | |
| 10 | PSD C | 500 nM ADMA/SDMA + 25 μM Arg | 1054 | 1060 | 100.6% |
| 11 | PSD C | 1000 nM ADMA/SDMA + 100 μM Arg | 1554 | 1610 | 103.6% |
| 12 | PSD C | 3000 nM ADMA/SDMA + 300 μM Arg | 3554 | 3840 | 108.0% |

| | | | Expected | Observed | |
|---|---|---|---|---|---|
| | | Arginine | Value | Value | % |
| Sample # | Sample ID | Recovery protocol | (μmol/L) | (μmol/L) | Recovery |
| 1 | PSD A | Water Spiked | | 68 | |
| 2 | PSD A | 500 nM ADMA/SDMA + 25 μM Arg | 93 | 98 | 106.0% |
| 3 | PSD A | 1000 nM ADMA/SDMA + 100 μM Arg | 168 | 175 | 104.4% |
| 4 | PSD A | 3000 nM ADMA/SDMA + 300 μM Arg | 368 | 346 | 94.1% |
| 5 | PSD B | Water Spiked | | 61 | |
| 6 | PSD B | 500 nM ADMA/SDMA + 25 μM Arg | 86 | 84 | 97.6% |
| 7 | PSD B | 1000 nM ADMA/SDMA + 100 μM Arg | 161 | 169 | 104.7% |
| 8 | PSD B | 3000 nM ADMA/SDMA + 300 μM Arg | 361 | 336 | 93.0% |
| 9 | PSD C | Water Spiked | | 91 | |
| 10 | PSD C | 500 nM ADMA/SDMA + 25 μM Arg | 116 | 135 | 116.6% |
| 11 | PSD C | 1000 nM ADMA/SDMA + 100 μM Arg | 191 | 197 | 103.2% |
| 12 | PSD C | 3000 nM ADMA/SDMA + 300 μM Arg | 391 | 361 | 92.4% |

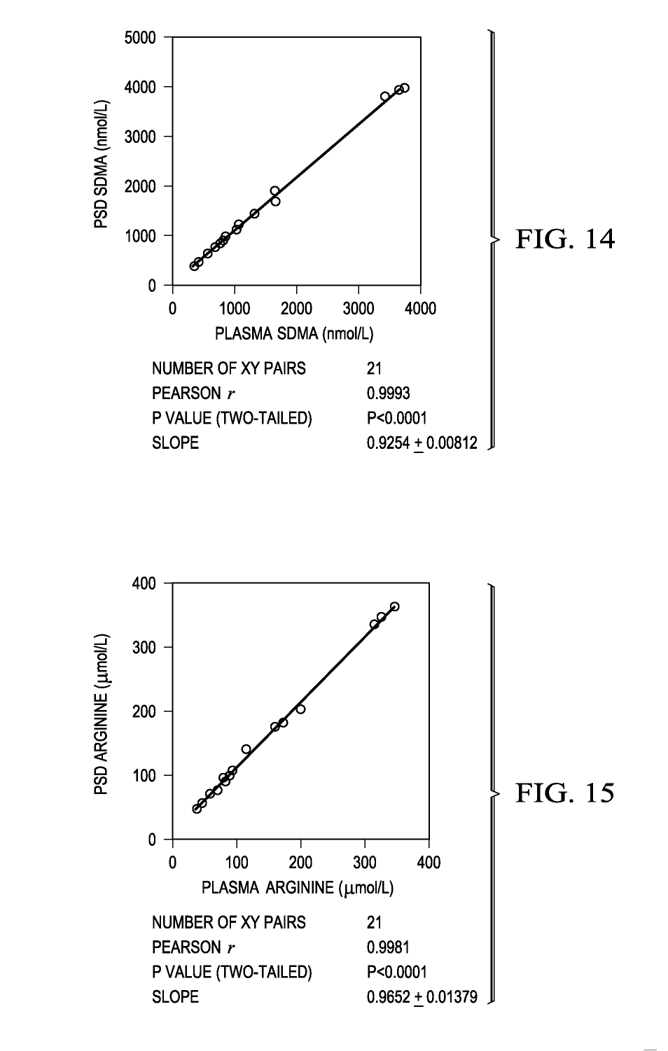

FIG. 13 is a graph that shows the correlation for ADMA in plasma versus PSD. FIG. 14 is a graph that shows the correlation for SDMA in plasma versus PSD. FIG. 15 is a graph that shows the correlation for Arginine in plasma versus PSD.

It was found that the collection of a blood sample on a PSD with subsequent filtration to obtain plasma is simple requiring a small volume that will result in fewer specimen rejections at the laboratory over traditional whole blood on filter paper. The PSD collection method allows accurate disease or treatment monitoring of subjects in a home setting or remote areas for clinical and/or research studies.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

C. D. M. van Karnebeek and S. Stockler, Treatable inborn errors of metabolism causing intellectual disability: A systematic literature review, Mol. Genetics and Metabolism, 105 (2012) 368-381.
Editorial, Asymmetric dimethylarginine (ADMA): Is really a biomarker for cardiovascular prognosis? Intl. Journal of Cardiology 153 (2011) 123-125.
A. Meinitzer, et al., Symmetrical and Asymmetrical Dimethylarginine as Predictors for Mortality in Patients Referred for Coronary Angiography: The Ludwigshafen Risk and Cardiovascular Health Study Clinical Chemistry 57:1 (2011) 112-121.
C. Wagner and M. Koury A-S-Adenosylhomocysteine—a better indicator of vascular disease than homocysteine? Am J Clin Nutr 2007; 86:1581-1585.
S. Stabler, et al., Elevation of Serum Cystathionine Levels in Patients with Cobalamin and Folate Deficiency Blood Vol 81, No 12 (1993) 3404-3413.
Physicians's Guide to the Laboratory Diagnosis of Metabolic Diseases, Blau, Duran and Blaskovics (Eds) (1996) Chapman and Hall, Alden Press Oxford, Chapter B, Amino Acid Analysis 24-28.
S. Stabler and R. Allen, Vitamin B12 Deficiency as a Worldwide Health Problem, Annu Rev. Nutr. (2004) 24:299-326.

What is claimed is:

1. A method of diagnosing and distinguishing one or more disorders from a single dried blood sample comprising the steps of:
obtaining a blood plasma sample from a plasma separator device, wherein the plasma separator device comprises a removable holding member to cover a semi-permeable blood separation member, a blood introducing portion formed in a portion of the holding member and in communication with the semi-permeable blood separation member, a removable plasma sample collection reservoir in communication with the semi-permeable blood separator member, wherein a whole blood sample is deposited on the blood introducing portion and separated by the semi-permeable blood separator member and the blood plasma sample is collected in the removable plasma sample collection reservoir, and a base in communication with the removable plasma sample collection reservoir; and
analyzing the plasma sample using a Liquid-chromatography-tandem-mass spectrometer (LC-MS/MS) to detect at least two analyte levels in the plasma sample to diagnose one or more disorders, wherein the at least two analyte levels are selected from total homocysteine, methylmalonic acid, s-adenosylhomocysteine, betaine, choline, asymmetric dimethylarginine, Symmetric dimethylarginine, creatinine, amino acids, glutathione, phenylalanine, tyrosine, Vitamin D, Vitamin B1 (thiamine), Vitamin B2 (riboflavin), Vitamin B3 (niacin), Vitamin B4 (adenine), Vitamin B5 (pantothenic acid), Vitamin B6 (pyridoxine), Vitamin B7 (biotin), Vitamin B12, folate or iron.

2. The method of claim 1, wherein the removable plasma sample collection reservoir is removed from the base.

3. The method of claim 2, wherein the plasma sample is isolated from the removable plasma sample collection reservoir.

4. The method of claim 1, further comprising the step of obtaining a white blood cell sample from the removable holding member.

5. The method of claim 1, further comprising the step of detecting 1, 2, 3, 4, 5, 6, 7, 8, 9, 10, 11, 12, 13, or 14 additional analyte levels selected from total homocysteine, methylmalonic acid, s-adenosylhomocysteine, betaine, choline, asymmetric dimethylarginine, Symmetric dimethylarginine, creatinine, amino acids, phenylalanine, tyrosine, Vitamin D, Vitamin B1 (thiamine), Vitamin B2 (riboflavin), Vitamin B3 (niacin), Vitamin B4 (adenine), Vitamin B5 (pantothenic acid), Vitamin B6 (pyridoxine), and Vitamin B7 (biotin).

6. The method of claim 1, wherein the step of receiving the plasma separator device is defined further as being received by mail.

7. The method of claim 1, wherein the one or more disorders are selected from at least one of a nutritional disorder, a hematological disease, a psychiatric disease, a neurological disease, a vascular disease, a peripheral disease, a cardiovascular disease, a cerebrovascular disease, a genetic metabolic disorder, a renal insufficiency, an Argininemia, an Argininosuccinic Aciduria, a Carbamoylphosphate Synthetase Deficiency1, a Citrullinemia, a Homocystinuria, a Hypermethioninemia, a Hyperammonemia, a Hyperornithinemia, a Homocitrullinuria, a Maple Syrup Urine Disease, a Phenylketonuria, a Tyrosinemia, a Cystathionine beta-synthease deficiency, a Methylenetetrahydrofolate reductase deficiency, or a Methylmalonic Acidemia.

8. A method of diagnosing a disease comprising the steps of:
obtaining a blood plasma sample from a blood plasma separator device, wherein the blood plasma separator device comprises a removable holding member to cover a semi-permeable blood separation member, a blood introducing portion formed in a portion of the holding member and in communication with the semi-permeable blood separation member, a removable blood plasma sample collection reservoir in communication with the semi-permeable blood separator member, wherein a whole blood sample is deposited on the blood introducing portion and separated by the semi-permeable blood separator member and a blood plasma sample is collected in the removable blood plasma sample collection reservoir, and a base in communication with the removable blood plasma sample collection reservoir; and
analyzing the blood plasma sample using a LC-MS/MS to detect at least two analyte levels in the blood plasma sample to diagnose one or more diseases, wherein the at least two analyte levels are selected from total homocysteine, methylmalonic acid, s-adenosylhomocysteine, betaine, choline, asymmetric dimethylarginine, symmetric dimethylarginine, creatinine, amino acids, glutathione, phenylalanine, tyrosine, Vitamin D, Vitamin B1 (thiamine), Vitamin B2 (riboflavin), Vitamin B3 (niacin), Vitamin B4 (adenine), Vitamin B5 (pantothenic acid), Vitamin B6 (pyridoxine), Vitamin B7 (biotin), Vitamin B12, folate, or iron.

9. The method of claim 8, further comprising the step of detecting 1, 2, 3, 4, 5, 6, 7, 8, 9, 10, 11, 12, 13, or 14 additional analyte levels selected from total homocysteine, methylmalonic acid, s-adenosylhomocysteine, betaine, choline, asymmetric dimethylarginine, Symmetric dimethylarginine, creatinine, an amino acids, glutathione, Vitamin D, Vitamin B1 (thiamine), Vitamin B2 (riboflavin), Vitamin B3 (niacin), Vitamin B4 (adenine), Vitamin B5 (pantothenic acid), Vitamin B6 (pyridoxine), Vitamin B7 (biotin), Vitamin B12, folate or iron.

10. The method of claim 8, wherein the analytes include total homocysteine, s-adenosylhomocysteine, asymmetric dimethylarginine, phenylalanine, and symmetric dimethylarginine and are used to diagnose one or more vascular risk factors

11. The method of claim 10, further comprising the step of detecting 1 or 2 additional analyte levels selected from total homocysteine, s-adenosylhomocysteine, asymmetric dimethylarginine, and symmetric dimethylarginine.

12. The method of claim 8, wherein the analytes include total homocysteine, Methionine, S-adenosylmethionine, S-adenosylhomocysteine, phenylalanine, and amino acids and are used to diagnose one or more genetic metabolic disorders.

13. The method of claim 8, further comprising the step of receiving the blood plasma separator device by mail.

14. The method of claim 8, wherein the analytes include at least two analyte levels are selected from S-adenosylhomocysteine, Asymmetric dimethylarginine, Symmetric dimethylarginine, and creatinine and are used to diagnose a renal insufficiency.

15. The method of claim 14, further comprising the step of detecting 1, 2, or 3 additional analyte levels selected from S-adenosylhomocysteine, Asymmetric dimethylarginine, Symmetric dimethylarginine, and creatinine.

16. The method of claim 8, wherein the analytes detected include the levels of total homocysteine and methylmalonic acid, wherein elevated levels of total homocysteine and methylmalonic acid indicate cobalamin deficiency and elevated levels of total homocysteine combined with normal levels of methylmalonic acid indicate folic acid deficiency to diagnose deficiency of cobalamin, folate, or both.

17. The method of claim 8, wherein the disease is selected from at least one of a nutritional disorder, a hematological disease, a psychiatric disease, a neurological disease, a vascular disease, a peripheral disease, a cardiovascular disease, a cerebrovascular disease, a genetic metabolic disorder, a renal insufficiency, an Argininemia, an Argininosuccinic Aciduria, a Carbamoylphosphate Synthetase Deficiency1, a Citrullinemia, a Homocystinuria, a Hypermethioninemia, a Hyperammonemia, a Hyperornithinemia, a Homocitrullinuria, a Maple Syrup Urine Disease, a Phenylketonuria, a Tyrosinemia, a Cystathionine beta-synthease deficiency, a Methylenetetrahydrofolate reductase deficiency, or a Methylmalonic Acidemia.

18. A method of monitoring a drug level in a subject in a clinical trial comprising the steps of:
(a) providing a subject involved in a trial;
(b) obtaining a blood plasma separator device from the subject;
(c) obtaining a blood plasma sample from the blood plasma separator device, wherein the blood plasma separator device comprises a removable holding member to cover a semi-permeable blood separation member, a blood introducing portion formed in a portion of the holding member and in communication with the semi-permeable blood separation member, a removable blood plasma sample collection reservoir in communication with the semi-permeable blood separator member, wherein a whole blood sample is deposited on the blood introducing portion and separated by the semi-permeable blood separator member and the blood plasma sample is collected in the removable blood plasma sample collection reservoir, and a base in communication with the removable blood plasma sample collection reservoir;
(d) analyzing the blood plasma sample using a LC-MS/MS to detect at least two analyte levels in the plasma sample, wherein the at least two analyte levels are selected from total Homocysteine (tHcy), Methylmalonic acid (MMA), S-adenosylhomocysteine (SAH), Betaine, Choline, Asymmetric dimethylarginine (ADMA), Symmetric dimethylarginine (SDMA), creatinine, Amino Acids, glutathione, phenylalanine, Vitamin D, Vitamin B1 (thiamine), Vitamin B2 (riboflavin), Vitamin B3 (niacin), Vitamin B4 (adenine), Vitamin B5 (pantothenic acid), Vitamin B6 (pyridoxine), Vitamin B7 (biotin), Vitamin B12, folate, or iron;
(e) providing an agent to the subject;
(f) analyzing the blood plasma sample using an LC-MS/MS to detect a agent level; and
(g) repeating steps (a) to (f).

19. The method of claim 18, wherein the clinical trial is for a nutritional disorder, a hematological disease, a psychiatric disease, a neurological disease, a vascular disease, a peripheral disease, a cardiovascular disease, a cerebrovascular disease, a genetic metabolic disorder, or a renal insufficiency.

20. The method of claim 18, wherein the clinical trial is pre-clinical trial and the subject is a cat, a dog, a goat, a non-human primate, a mouse, a pig, or a rat.

21. The method of claim 18, wherein the clinical trial is clinical drug trial and the subject is a human.

22. A system for diagnosing and distinguishing multiple disorders from a single dried blood sample comprising:
a blood plasma separator comprising a removable holding member to cover a semi-permeable blood separation member, a blood introducing portion formed in a portion of the holding member and in communication with the semi-permeable blood separation member, a removable blood plasma sample collection reservoir in communication with the semi-permeable blood separator member, wherein a whole blood sample deposited on the blood introducing portion is separated by the semi-permeable blood separator member and the blood plasma sample is collected in the removable blood plasma sample collection reservoir, and a base in communication with the removable blood plasma sample collection reservoir; and
a LC-MS/MS system to detect at least two analyte levels in the blood plasma sample to diagnose multiple disorders and distinguishing therebetween, wherein the at least two analyte levels are selected from total Homocysteine (tHcy), Methylmalonic acid (MMA), S-adenosylhomocysteine (SAH), Betaine, Choline, Asymmetric dimethylarginine (ADMA), Symmetric dimethylarginine (SDMA), creatinine, Amino Acids, glutathione, phenylalanine, Vitamin D, Vitamin B1 (thiamine), Vitamin B2 (riboflavin), Vitamin B3 (niacin), Vitamin B4 (adenine), Vitamin B5 (pantothenic acid), Vitamin B6 (pyridoxine), Vitamin B7 (biotin), Vitamin B12, folate, or iron.

23. A method of multiplex sample analysis from a single dried blood sample comprising the steps of:
obtaining a blood plasma sample from a plasma separator device, wherein the plasma separator device comprises a removable holding member to cover a semi-permeable blood separation member, a blood introducing portion formed in a portion of the holding member and in communication with the semi-permeable blood separation member, a removable plasma sample collection reservoir in communication with the semi-permeable blood separator member, wherein a whole blood sample is deposited on the blood introducing portion and separated by the semi-permeable blood separator member and the blood plasma sample is collected in the removable plasma sample collection reservoir, and a base in communication with the removable plasma sample collection reservoir;
labeling one or more components of the plasma sample; and
analyzing the plasma sample using a liquid-chromatography-tandem-mass spectrometer (LC-MS/MS) to detect the one or more components in the plasma sample.

24. A blood plasma separator comprising:
a removable holding member to cover a semi-permeable blood separation member,
a blood introducing portion formed in a portion of the holding member and in communication with the semi-permeable blood separation member,
a removable blood plasma sample collection reservoir in communication with the semi-permeable blood separator member, wherein a whole blood sample deposited on the blood introducing portion is separated by the semi-permeable blood separator member and the blood plasma sample is collected in the removable blood plasma sample collection reservoir, and
a base in communication with the removable blood plasma sample collection reservoir.

25. A method of monitoring a drug level in a subject comprising the steps of:
(a) obtaining a blood plasma separator device from the subject;
(b) obtaining a blood plasma sample from the blood plasma separator device, wherein the blood plasma separator device comprises a removable holding member to cover a semi-permeable blood separation member, a blood introducing portion formed in a portion of the holding member and in communication with the semi-permeable blood separation member, a removable blood plasma sample collection reservoir in communication with the semi-permeable blood separator member, wherein a whole blood sample is deposited on the blood introducing portion and separated by the semi-permeable blood separator member and the blood plasma sample is collected in the removable blood plasma sample collection reservoir, and a base in communication with the removable blood plasma sample collection reservoir;
(c) analyzing the blood plasma sample using a LC-MS/MS to detect at least two analyte levels in the plasma sample, wherein the at least two analyte levels are selected from total Homocysteine (tHcy), Methylmalonic acid (MMA), S-adenosylhomocysteine (SAH), Betaine, Choline, Asymmetric dimethylarginine (ADMA), Symmetric dimethylarginine (SDMA), creatinine, Amino Acids, glutathione, phenylalanine, Vitamin D, Vitamin B1 (thiamine), Vitamin B2 (riboflavin), Vitamin B3 (niacin), Vitamin B4 (adenine), Vitamin B5 (pantothenic acid), Vitamin B6 (pyridoxine), Vitamin B7 (biotin), Vitamin B12, folate, or iron;
(d) providing an agent to the subject;
(e) analyzing the blood plasma sample using an LC-MS/MS to detect the level of the agent; and
(f) optionally repeating steps (a) to (e), if necessary.

26. The method of claim 25, wherein the disease is selected from at least one of a nutritional disorder, a hematological disease, a psychiatric disease, a neurological disease, a vascular disease, a peripheral disease, a cardiovascular disease, a cerebrovascular disease, a genetic metabolic disorder, a renal insufficiency, an Argininemia, an Argininosuccinic Aciduria, a Carbamoylphosphate Synthetase Deficiency1, a Citrullinemia, a Homocystinuria, a Hypermethioninemia, a Hyperammonemia, a Hyperornithinemia, a Homocitrullinuria, a Maple Syrup Urine Disease, a Phenylketonuria, a Tyrosinemia, a Cystathionine beta-synthease deficiency, a Methylenetetrahydrofolate reductase deficiency, or a Methylmalonic Acidemia.
